(19) United States
(12) Patent Application Publication
(10) Pub. No.: US 2025/0234767 A1
(43) Pub. Date: Jul. 17, 2025

(54) INITIATOR, QUANTUM DOT LIGHT-EMITTING LAYER AND MANUFACTURING METHOD THEREOF, LIGHT-EMITTING DEVICE, DISPLAY DEVICE

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Bing FANG, Beijing (CN); Shaoyong LU, Beijing (CN); Zhuo CHEN, Beijing (CN); Yichi ZHANG, Beijing (CN)

(21) Appl. No.: 18/703,765

(22) PCT Filed: Aug. 8, 2023
(86) PCT No.: PCT/CN2023/111653

§ 371 Date: Apr. 23, 2024

(30) Foreign Application Priority Data
Mar. 23, 2023 (WO) PCT/CN2023/083509

(51) Int. Cl. H10K 85/10; C07D 295/112; C09K 11/06; H10K 50/115; H10K 71/13

(52) U.S. Cl. CPC H10K 85/141; C07D 295/112; C09K 11/06; C09K 2211/14; H10K 50/115; H10K 71/13

(57) ABSTRACT

An initiator, a quantum dot, a quantum dot light-emitting layer and a manufacturing method thereof, a light-emitting device, a display device. The structural formula of the initiator is

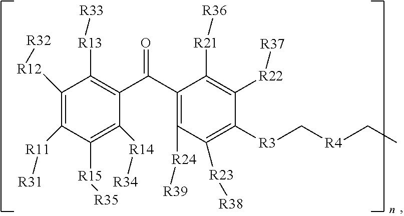

R11˜R15 are any one of H, a group including N and H, ester, alkyl; R21˜R24 are any one of H, F, S, O, ester, alkyl, a group including N and H; R3 is any one of S, O, ester, amide, alkyl, H, F; R4 is any one of ester, alkyl, a group including N and H; R31˜R39 are present or absent, when at least one of R31˜R39 is present, it is any one of ester, alkyl, a group including N and H; n≥1 and is a positive integer, a group of the initiator includes at least one of S, O, the group including N and H, the group of the initiator refers to a group except a benzophenone matrix.

### RELATED APPLICATIONS

This application claims the priority of PCT International Application No. PCT/CN2023/083509 filed on Mar. 23, 2023, the entire disclosure of which is incorporated herein by reference.

### TECHNICAL FIELD

The present disclosure relates to the field of display technology, and in particular, to an initiator, a quantum dot, a quantum dot light-emitting layer, a light-emitting device, a display device, and a method of manufacturing a patterned quantum dot light-emitting layer.

### BACKGROUND

Light Emitting Diode (LED) emits light by releasing energy through the recombination of electrons and holes. The light emitting diode comprises, but is not limited to, an organic light emitting diode (OLED) and a quantum dot light emitting diode (QLED). Quantum dots are semiconductor nanomaterials that can trap excitons in three dimensions. Due to excellent properties such as high quantum efficiency, narrow excitation spectrum, high stability of light, long fluorescence lifetime, and good solution processing compatibility, the quantum dots have huge application potential in high-quality display. The quantum dot light emitting diode is a device that uses quantum dots as light-emitting materials. Compared with the organic light emitting diode, the quantum dot light emitting diode has outstanding advantages such as lower energy consumption, higher color purity, and wider color gamut, so the quantum dot light emitting technology has become the most promising next-generation self-light-emitting display technology.

### SUMMARY

According to an aspect of the present disclosure, an initiator is provided. The structural formula of the initiator is

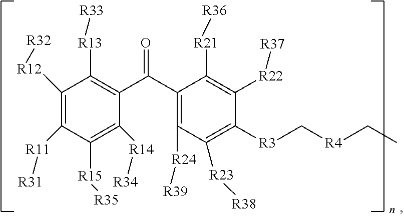

where R11, R12, R13, R14 and R15 are same or different from each other, and R11, R12, R13, R14 and R15 are respectively selected from any one of a hydrogen atom, a group comprising a nitrogen atom and a hydrogen atom, an ester group, and an alkyl group; R21, R22, R23 and R24 are same or different from each other, and R21, R22, R23 and R24 are respectively selected from any one of a hydrogen atom, a fluorine atom, a sulfur atom, an oxygen atom, an ester group, an alkyl group, and a group comprising a nitrogen atom and a hydrogen atom; R3 is selected from any one of a sulfur atom, an oxygen atom, an ester group, an amide group, an alkyl group, a hydrogen atom, and a fluorine atom; R4 is selected from any one of an ester group, an alkyl group, and a group comprising a nitrogen atom and a hydrogen atom; R31, R32, R33, R34, R35, R36, R37, R38 and R39 are present or absent and are same or different from each other, when at least one of R31, R32, R33, R34, R35, R36, R37, R38 and R39 is present, the at least one of R31, R32, R33, R34, R35, R36, R37, R38 and R39 is selected from any one of an ester group, an alkyl group, and a group comprising a nitrogen atom and a hydrogen atom; and n is a positive integer greater than or equal to 1, a group of the initiator comprises at least one of the sulfur atom, the oxygen atom, and the group comprising the nitrogen atom and the hydrogen atom, the group of the initiator refers to a group except a benzophenone matrix.

In some embodiments, R31, R32, R33, R34, R35, R36, R37, R38 and R39 are absent, R11, R12, R13, R14 and R15 are respectively selected from the hydrogen atom or the group comprising the nitrogen atom and the hydrogen atom, R21, R22, R23 and R24 are respectively selected from the hydrogen atom or the fluorine atom, and R3 is selected from any one of the sulfur atom, the oxygen atom, the ester group, the amide group, and the alkyl group.

In some embodiments, the group comprising the nitrogen atom and the hydrogen atom is a pyrrole group or a tertiary amine group.

In some embodiments, the structural formula of the initiator is

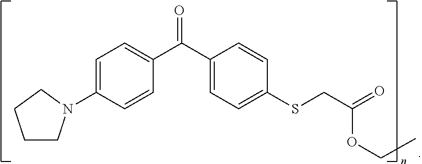

In some embodiments, n=3, the structural formula of the initiator is

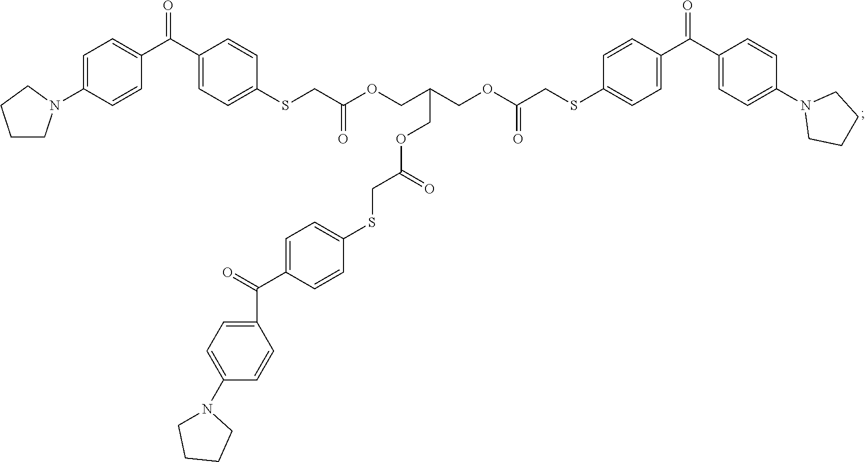 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(=O)c2ccc(N3CCCC3)cc2)cc1]

or,
n=2, the structural formula of the initiator is

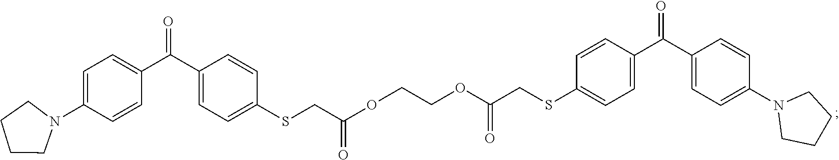 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]

or,
n=4, the structural formula of the initiator is

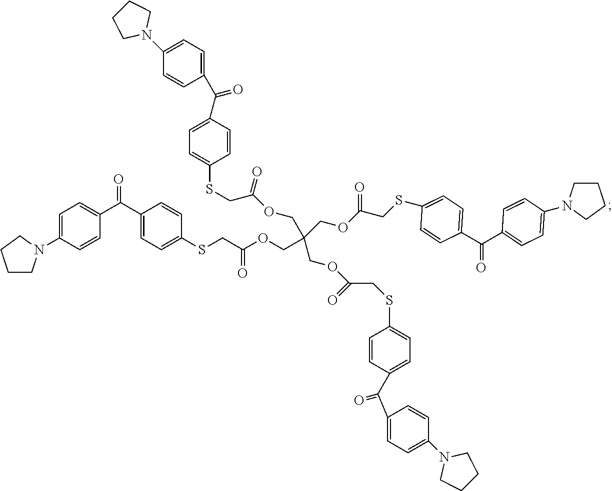 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)(COC(=O)CSc1ccc(C(=O)c2ccc(N3CCCC3)cc2)cc1)COC(=O)CSc1ccc(C(=O)c2ccc(N3CCCC3)cc2)cc1]

or,
n=1, the structural formula of the initiator is

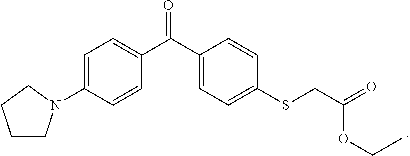 [SMILES: CCOC(=O)CSc1ccc(C(=O)c2ccc(N3CCCC3)cc2)cc1]

In some embodiments, the structural formula of the initiator is

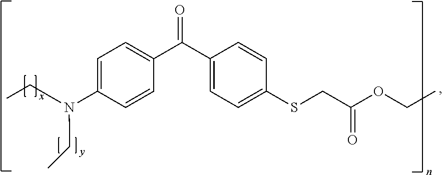

where x and y are both positive integers greater than or equal to 1.

In some embodiments, a number of carbon atoms in a branch chain connected to the nitrogen atom is 2˜30.

According to another aspect of the present disclosure, an initiator is provided. The initiator is a mixture of a benzophenone and an amine compound or a mixture of a benzophenone derivative and an amine compound. A structural formula of the benzophenone derivative is

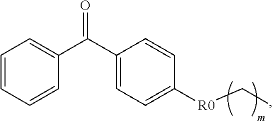

R0 is selected from any one of O, S, C₆H₅, OH, Br, Cl, and I, and m is a positive integer greater than or equal to 0.

In some embodiments, the amine compound is a pyrrole or a tertiary amine.

According to yet another aspect of the present disclosure, a quantum dot is provided. A surface of the quantum dot has a ligand, and a structural formula of the ligand is

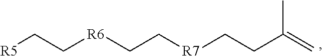

where R5 is coordinately connected to the surface of the quantum dot and is selected from any one of a sulfhydryl group, a carboxyl group and an amino group, R6 is selected from an ester group or an ether group, R7 is selected from an ester group or an ether group, the ligand is configured to undergo a photosensitive reaction under light radiation with the initiator described in any of the previous embodiments.

In some embodiments, the structural formula of the ligand is

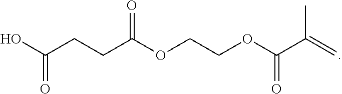 [SMILES: C=C(C)C(=O)OCCOC(=O)CCC(=O)O]

According to still another aspect of the present disclosure, a quantum dot light-emitting layer is provided. The quantum dot light-emitting layer comprises a plurality of quantum dots. A surface of at least some of the plurality of quantum dots has a ligand, and the quantum dot light-emitting layer is generated by crosslinking the quantum dots having the ligands with the initiator described in any of the previous embodiments.

In some embodiments, a structural formula of the ligand is

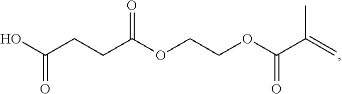 [SMILES: C=C(C)C(=O)OCCOC(=O)CCC(=O)O]

the structural formula of the initiator is

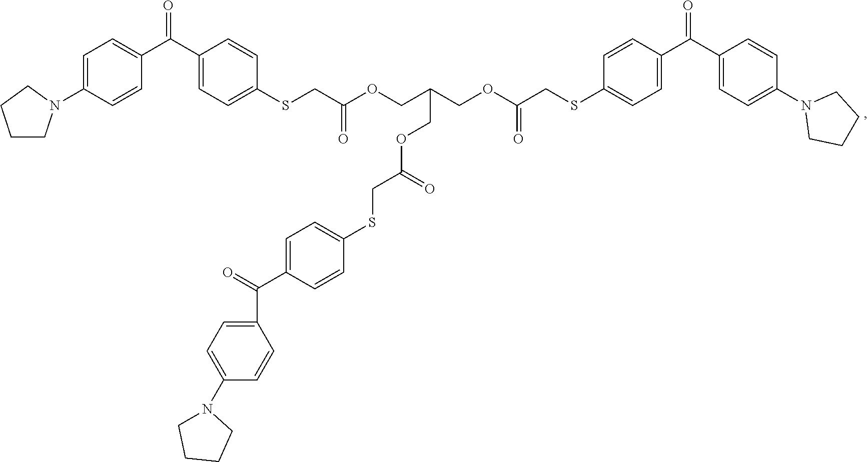 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(=O)c2ccc(N3CCCC3)cc2)cc1]

and a structural formula of the quantum dot light-emitting layer is

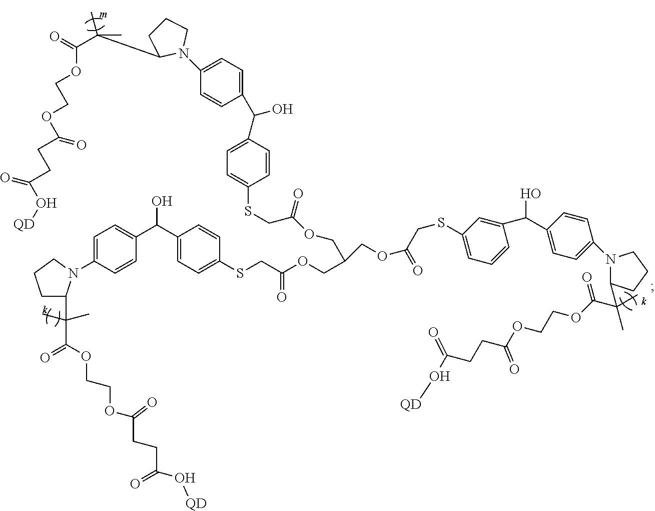

or,
a structural formula of the ligand is

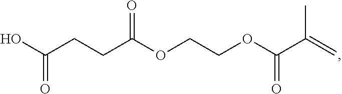 [SMILES: C=C(C)C(=O)OCCOC(=O)CCC(=O)O]

the structural formula of the initiator is

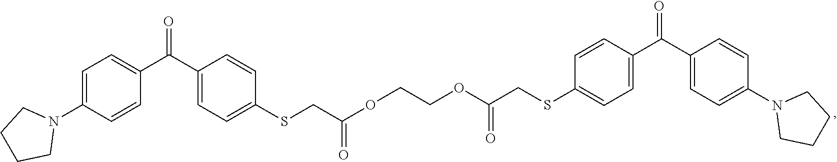 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]

and a structural formula of the quantum dot light-emitting layer is

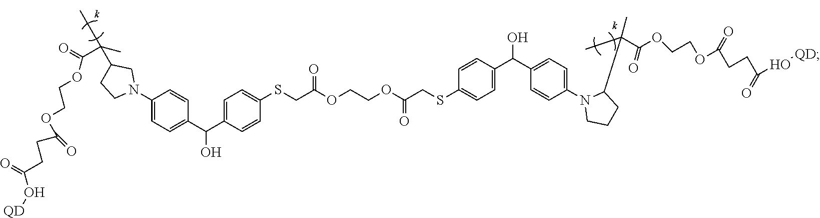

or,
a structural formula of the ligand is

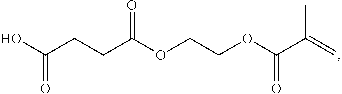 [SMILES: C=C(C)C(=O)OCCOC(=O)CCC(=O)O]

the structural formula of the initiator is

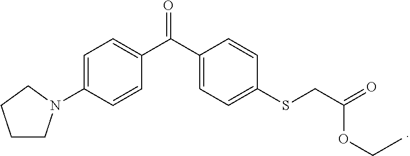 [SMILES: CCOC(=O)CSc1ccc(C(=O)c2ccc(N3CCCC3)cc2)cc1]

and a structural formula of the quantum dot light-emitting layer is

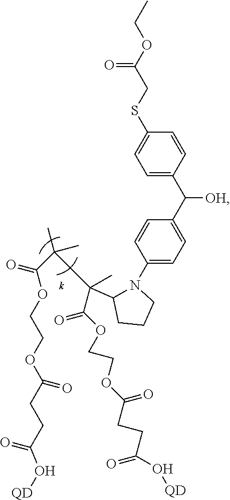

where QD represents a quantum dot, and k is a positive integer greater than or equal to 1.

In some embodiments, a structural formula of the ligand is

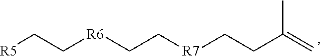

the initiator is a mixture of a benzophenone and an amine compound, and a structural formula of the quantum dot light-emitting layer is

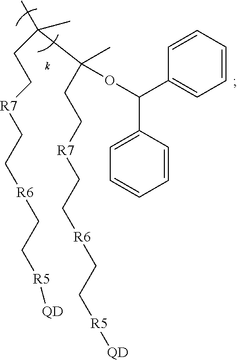

or,
a structural formula of the ligand is

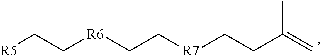

the initiator is a mixture of a benzophenone derivative and an amine compound, a structural formula of the benzophenone derivative is

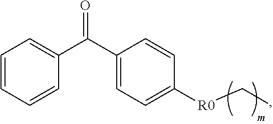

and a structural formula of the quantum dot light-emitting layer is

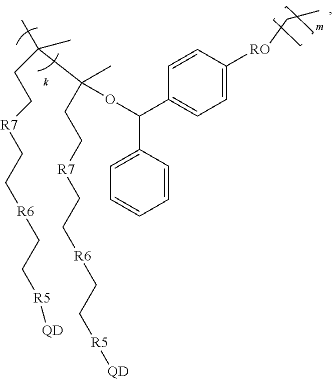

where R5 is selected from any one of a sulfhydryl group, a carboxyl group and an amino group, R6 is selected from an ester group or an ether group, R7 is selected from an ester group or an ether group, R0 is selected from any one of O, S, C₆H₅, OH, Br, Cl and I, QD represents a quantum dot, k is a positive integer greater than or equal to 1, and m is a positive integer greater than or equal to 0.

In some embodiments, the quantum dot light-emitting layer comprises a plurality of first quantum dot patterns, a plurality of second quantum dot patterns, and a plurality of third quantum dot patterns, the first quantum dot patterns are configured to emit red light, the second quantum dot patterns are configured to emit green light, and the third quantum dot patterns are configured to emit blue light.

According to yet another aspect of the present disclosure, a light emitting device is provided. The light-emitting device comprises: a first electrode layer; a hole injection layer on the first electrode layer; a hole transport layer on a side of the hole injection layer away from the first electrode layer; the quantum dot light-emitting layer described in any of the previous embodiments, the quantum dot light-emitting layer being on a side of the hole transport layer away from the first electrode layer; an electron transport layer on a side of the quantum dot light-emitting layer away from the first electrode layer; and a second electrode layer on a side of the electron transport layer away from the first electrode layer.

According to yet another aspect of the present disclosure, a display device is provided. The display device comprises a plurality of light-emitting devices described in any of the previous embodiments, at least two of the plurality of light-emitting devices are configured to emit light of different colors.

According to yet another aspect of the present disclosure, a method of manufacturing a patterned quantum dot light-emitting layer is provided. The method comprises: providing a substrate; applying a mixed solution on the substrate, the mixed solution comprising a quantum dot and an initiator, a surface of the quantum dot having a ligand comprising a carbon-carbon double bond, and the initiator being the initiator described in any of the previous embodiments; and initiating a polymerization reaction of the carbon-carbon double bonds of the ligands by the initiator under light radiation to form the patterned quantum dot light-emitting layer.

In some embodiments, the method further comprises: curing the mixed solution applied on the substrate to form an intermediate layer. The initiating the polymerization reaction of the carbon-carbon double bonds of the ligands by the initiator under light radiation to form the patterned quantum dot light-emitting layer, comprises: exposing the intermediate layer by using a mask, allowing ultraviolet light to pass through the mask to expose the intermediate layer, and initiating the polymerization reaction of the carbon-carbon double bonds of the ligands by the initiator under the ultraviolet light; and developing the polymerized intermediate layer by using a developer, and dissolving an unexposed portion of the intermediate layer by the developer to form the patterned quantum dot light-emitting layer.

In some embodiments, a concentration of the quantum dots in the mixed solution is about 25˜30 mg/mL, and a concentration of the initiator in the mixed solution is about 0.1˜1.0 mg/mL.

### BRIEF DESCRIPTION OF THE DRAWINGS

Various exemplary embodiments of the present disclosure will be described in detail below with reference to the accompanying drawings, in which:

FIG. 1 illustrates a schematic diagram of the mechanism of benzophenone initiating polymerization of free radicals under light radiation;

FIG. 2 illustrates a schematic diagram of polymerization of carbon-carbon double bonds initiated by free radicals;

FIG. 3 illustrates a structural formula of an initiator according to an embodiment of the present disclosure;

FIG. 4 illustrates a mechanism diagram of a tertiary amine inhibiting polymerization inhibition of triplet benzophenone structural unit by oxygen;

FIG. 5 illustrates a photosensitivity mechanism diagram of a benzophenone structural unit comprising a sulfur atom;

FIG. 6 illustrates a schematic diagram of the molecular structure of an initiator with multiple initiating sites according to an embodiment of the present disclosure;

FIG. 7 illustrates a structural formula of an initiator according to an embodiment of the present disclosure;

FIG. 8 illustrates the synthesis steps of the intermediate product of the initiator SBP according to an embodiment of the present disclosure;

FIG. 9 illustrates the synthesis steps of the initiator SBP according to an embodiment of the present disclosure;

FIG. 10 illustrates a hydrogen nuclear magnetic resonance spectrum of the initiator SBP according to an embodiment of the present disclosure;

FIG. 11 illustrates a mass spectrum of the initiator SBP according to an embodiment of the present disclosure;

FIG. 12 illustrates a hydrogen nuclear magnetic resonance spectrum of SBP—COOH according to an embodiment of the present disclosure;

FIG. 13 illustrates a mass spectrum of SBP—COOH according to an embodiment of the present disclosure;

FIG. 14 illustrates a structural formula of an initiator (SBP)₂ according to an embodiment of the present disclosure;

FIG. 15 illustrates a hydrogen nuclear magnetic resonance spectrum of the initiator (SBP)₂ according to an embodiment of the present disclosure;

FIG. 16 illustrates a mass spectrum of the initiator (SBP)₂ according to an embodiment of the present disclosure;

FIG. 17 illustrates a structural formula of an initiator (SBP)₃ according to an embodiment of the present disclosure;

FIG. 18 illustrates a hydrogen nuclear magnetic resonance spectrum of the initiator (SBP)₃ according to an embodiment of the present disclosure;

FIG. 19 illustrates a mass spectrum of the initiator (SBP)₃ according to an embodiment of the present disclosure;

FIG. 20 illustrates the ultraviolet absorption spectra of initiators SBP, (SBP)₂, and (SBP)₃ according to embodiments of the present disclosure;

FIG. 21 illustrates a schematic diagram of a molecular structure of an initiator comprising multiple initiating sites according to an embodiment of the present disclosure;

FIG. 22 illustrates a structural formula of an initiator according to an embodiment of the present disclosure;

FIG. 23 illustrates a structural formula of an initiator according to an embodiment of the present disclosure;

FIG. 24 illustrates the synthesis steps of an initiator according to an embodiment of the present disclosure;

FIG. 25 illustrates a schematic diagram of the principle of increasing the initiation rate using a mixture of benzophenone and amine compounds provided according to an embodiment of the present disclosure;

FIG. 26 illustrates a schematic diagram of the principle of increasing the initiation rate by using a mixture of a benzophenone derivative and an amine compound provided according to an embodiment of the present disclosure;

FIG. 27 illustrates a structural formula of a ligand on a surface of the quantum dot according to an embodiment of the present disclosure;

FIG. 28 illustrates a flow chart of a method of preparing a patterned quantum dot light-emitting layer according to an embodiment of the present disclosure;

FIG. 29 illustrates a schematic diagram of a method of preparing a patterned quantum dot light-emitting layer according to an embodiment of the present disclosure;

FIG. 30 illustrates a schematic diagram of the mechanism by which the initiator SBP initiates the polymerization of ligands of the quantum dots according to an embodiment of the present disclosure;

FIG. 31 illustrates a schematic diagram of the mechanism by which the initiator (SBP)₂ initiates the polymerization of ligands of the quantum dots according to an embodiment of the present disclosure;

FIG. 32 illustrates a schematic diagram of the mechanism by which the initiator (SBP)₃ initiates the polymerization of ligands of the quantum dots according to an embodiment of the present disclosure;

FIG. 33 illustrates electron paramagnetic resonance spectra of quantum dot light-emitting layers respectively comprising initiators SBP, (SBP)₂, (SBP)₃ according to an embodiment of the present disclosure;

FIG. 34 illustrates the effect of exposure doses of different initiators SBP, (SBP)₂, (SBP)₃ on the film retention rate of the quantum dot light-emitting layer according to an embodiment of the present disclosure;

FIG. 35 illustrates a scanning electron microscope picture and an atomic force microscope picture of a quantum dot light-emitting layer according to an embodiment of the present disclosure;

FIG. 36 illustrates the relationship between the concentration of the initiator (SBP)₃ and the thickness of the quantum dot light-emitting layer according to an embodiment of the present disclosure;

FIG. 37 illustrates pictures under a fluorescence microscope of quantum dot light-emitting layers comprising initiators (SBP)₃ with different concentrations according to an embodiment of the present disclosure;

FIG. 38 illustrates pictures of patterned red, green, and blue quantum dot light-emitting layers on a backplane with different pixel densities according to an embodiment of the present disclosure;

FIG. 39 illustrates the effect of the concentration of the initiator (SBP)₃ on the current efficiency of the light-emitting device according to an embodiment of the present disclosure;

FIG. 40 illustrates the effect of the concentration of the initiator (SBP)₃ on the external quantum efficiency of the light-emitting device according to an embodiment of the present disclosure;

FIG. 41 illustrates a structural diagram of a light-emitting device according to an embodiment of the present disclosure;

FIG. 42 illustrates fluorescence microscope pictures of a quantum dot light-emitting layer according to an embodiment of the present disclosure;

FIG. 43 illustrates spectra of light-emitting devices under electrical excitation according to an embodiment of the present disclosure; and

FIG. 44 illustrates a block diagram of a display device according to an embodiment of the present disclosure.

It should be understood that the accompanying drawings are merely schematic illustrations of exemplary embodiments of the present disclosure, are not limitations of the present disclosure, and are not necessarily drawn to scale. Furthermore, in the accompanying drawings, the same or similar components are designated with the same or similar reference numerals.

### DETAILED DESCRIPTION OF THE DISCLOSURE

The technical solutions in the embodiments of the present disclosure will be clearly described below with reference to the accompanying drawings. Obviously, the described embodiments are only some, but not all, of the embodiments of the present disclosure. Based on the embodiments in the present disclosure, all other embodiments obtained by those of ordinary skill in the art without undue experimentation fall within the scope of protection of the present disclosure.

Before formally describing the technical solutions of the embodiments of the present disclosure, the terms used in the embodiments of the present disclosure are explained and defined as follows to help those skilled in the art understand the technical solutions of the embodiments of the present disclosure more clearly.

As used herein, the term “initiator” refers to a type of compounds that readily decompose into free radicals under some conditions, such as light radiation, which can be used to initiate polymerization and copolymerization of free radicals of alkene monomer and diene monomer, and can also be used for cross-link and curing of unsaturated polyesters and cross-link of polymers.

As used herein, the term “tertiary amine” or “tertiary amine group” refers to a molecule or group in which a trivalent amine is connected to three hydrocarbons. The general structural formula of tertiary amine can be expressed as

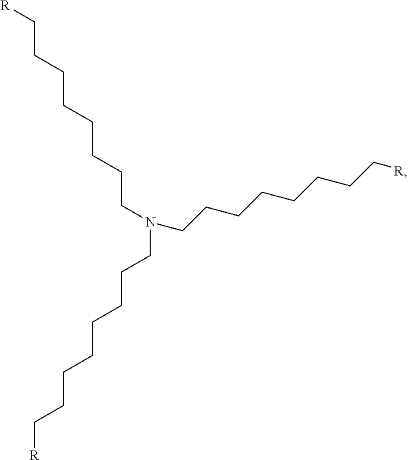

where the hydrocarbon R may have any suitable number of carbon atoms.

Quantum dot light-emitting diode display is a new display technology developed on the basis of organic light-emitting diode display. The quantum dot light-emitting diode display uses the quantum dot light-emitting layer as the light-emitting layer. The principle of emitting light of the quantum dot light-emitting diode display is to inject holes and electrons into the quantum dot light-emitting layer through the hole transport layer and the electron transport layer respectively, and the holes and electrons recombine in the quantum dot light-emitting layer to achieve light emission. Compared with the organic light-emitting diode display, the quantum dot light-emitting diode display has the advantages of higher color saturation, wider color gamut, narrower light-emitting peak and better stability. With the in-depth development of quantum dot technology, the research based on quantum dot display has become increasingly in-depth, and with the continuous improvement of quantum efficiency, the quantum dot light-emitting diode display has basically reached the level of industrialization. Therefore, it has become a future trend to develop new processes and technologies for the preparation of the quantum dot light-emitting layer to make the preparation process of the quantum dot light-emitting diode display more excellent.

The traditional method of patterning the quantum dot light-emitting layer is to expose and develop the photoresist layer to form a patterned photoresist. Then, the patterned photoresist is used to block the quantum dot light-emitting layer, and the unnecessary portions are etched, thereby forming a patterned quantum dot light-emitting layer. Although the traditional photoresist method can realize the patterning of quantum dots, the further application of this traditional method is limited due to problems such as complex process and poor solvent compatibility. To address this limitation, there is an urgent need to develop a new method of patterning quantum dots.

Recently, the industry has proposed that the quantum dot film may be directly exposed and developed to form a patterned quantum dot light-emitting layer without the use of photoresist. Such a manufacturing method is simple and low-cost, and can obtain a patterned quantum dot light-emitting layer with high quality, thereby realizing a quantum dot light-emitting diode display device with higher resolution. When direct photolithography is performed on the quantum dot light-emitting layer to achieve patterning, an initiator needs to be added to the quantum dot solution to cause a photosensitive reaction between the initiator and the ligands on the surface of the quantum dot, so as to form the patterned quantum dot light-emitting layer. The reactivity between the initiator and the ligands on the surface of the quantum dot determines the stability of the patterned quantum dot light-emitting layer. Therefore, the development of initiators with highly activity is crucial for the method of directly patterning the quantum dot light-emitting layer.

In the related art, benzophenone can be used as an initiator. FIG. 1 illustrates a schematic diagram of the mechanism of benzophenone initiating polymerization of free radicals under light radiation. As illustrated in FIG. 1, the benzophenone transitions to a singlet excited state after being radiated, and then undergoes an internal transition to a more stable triplet excited state. Subsequently, the triplet excited benzophenone can take away the hydrogen atom on the molecular chain PH, causing the molecular chain PH to become a free radical P·, and the benzophenone that takes away the hydrogen atom becomes semibenzene pinacol

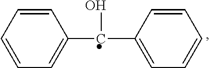

abbreviated as K·. Two semibenzene pinacols K· can be combined to form benzene-pinacol K—K, which is the photolysis product of benzophenone; at the same time, the two free radicals P· generated by the reaction can also form a cross-link bond P—P.

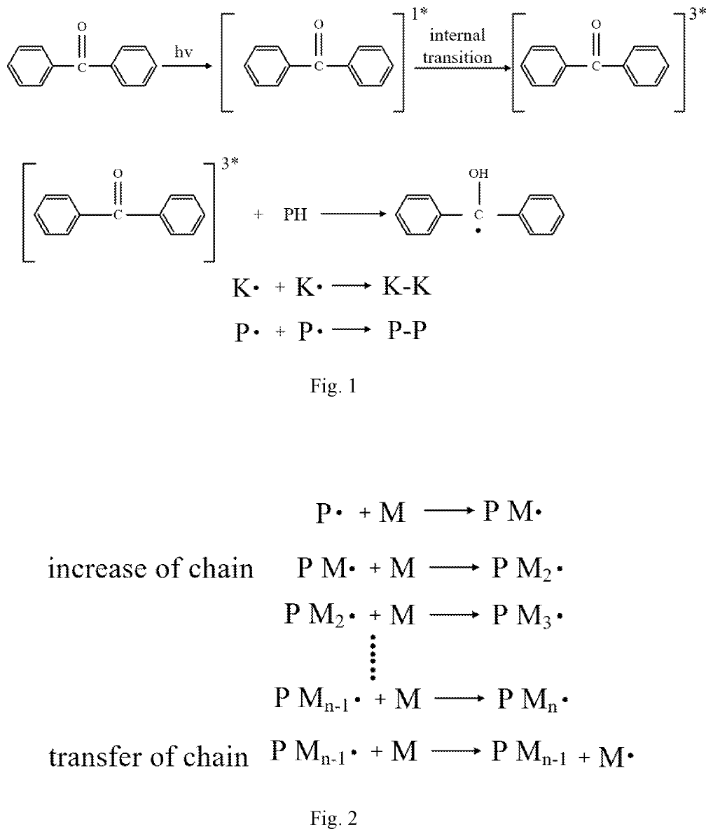

In addition, P· radical can also initiate polymerization of free radicals of carbon-carbon double bonds. As illustrated in FIG. 2, the activity of P· radical is transferred to the monomer M to form the monomer radical M·. By analogy, the free radical M· continuously initiates monomers to form a polymer. As the reaction proceeds, the chain length increases, and the activity of the monomer free radical decreases, resulting in chain transfer to transfer the activity to the monomer. A new active center is formed, thereby achieving chain growth.

However, the inventor(s) of the present application found that the polymerization rate of monomers initiated by the benzophenone is relatively slow, this may be because the triplet benzophenone is easily quenched by oxygen in the air, thereby weakening the polymerization activity. Therefore, using benzophenone as an initiator to initiate the polymerization reaction of ligands on the surface of quantum dots causes low initiation rate, which is not conducive to improving production efficiency, resulting in a significant increase in production costs.

In view of this, some embodiments of the present disclosure provide a type of initiators that can improve the activity and stability of free radicals, significantly improve the initiating efficiency, contribute to efficiently form a patterned quantum dot light-emitting layer with high quality, improve the production efficiency, and reduce the production costs.

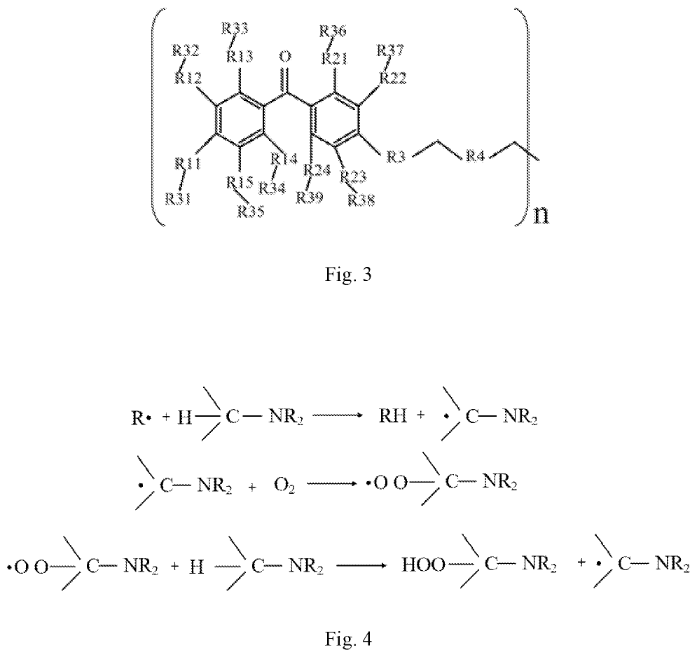

FIG. 3 illustrates a structural formula of an initiator provided according to embodiments of the present disclosure, the structural formula of the initiator is

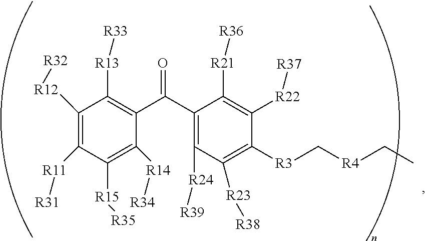

which is hereinafter abbreviated as a structural formula (1). In the structural formula (1), R11, R12, R13, R14, and R15 may be same or different from each other, and R11, R12, R13, R14, and R15 may be respectively selected from any one of a hydrogen atom, a group comprising a nitrogen atom and a hydrogen atom, an ester group, and an alkyl group; R21, R22, R23 and R24 mat be same or different from each other, and R21, R22, R23, and R24 may be respectively selected from any one of a hydrogen atom, a fluorine atom, a sulfur atom, an oxygen atom, an ester group, an alkyl group, and a group comprising a nitrogen atom and a hydrogen atom; R3 may be selected from any one of a sulfur atom, an oxygen atom, an ester group, an amide group, an alkyl group, a hydrogen atom and a fluorine atom; R4 may be selected from any one of an ester group, an alkyl group, and a group comprising a nitrogen atom and a hydrogen atom; R31, R32, R33, R34, R35, R36, R37, R38, and R39 may be present or absent and may be same or different from each other, when at least one of R31, R32, R33, R34, R35, R36, R37, R38, and R39 is present, the at least one of R31, R32, R33, R34, R35, R36, R37, R38, and R39 may be selected from any one of an ester group, an alkyl group, and a group comprising a nitrogen atom and a hydrogen atom; n is a positive integer greater than or equal to 1, and a group of the initiator comprises at least one of the sulfur atom, the oxygen atom, and the group comprising the nitrogen atom and the hydrogen atom. It should be noted that the phrase “a group of the initiator” refers to other groups except the benzophenone matrix, that is, it refers to any one or more of R11˜R15, R21˜R24, R31˜R39, R3, and R4.

It should be noted that in the structural formula (1), the types of groups of R11˜R15, R21˜R24, R31˜R39, R3, and R4 may be flexibly combined according to the types described above, as long as ensuring that the group of the initiator comprises at least one of the sulfur atom, the oxygen atom, and the group comprising the nitrogen atom and the hydrogen atom.

The reason why the initiator can increase the initiation rate will be explained below.

The initiator is further designed and optimized on the basis of the molecular structure of benzophenone to form the structural formula (1) as described above. As mentioned before, benzophenone eventually transitions to a more stable triplet excited state after being radiated, but the triplet benzophenone is easily quenched by oxygen in the air. However, in the structural formula (1), the group of the initiator comprises at least one of a sulfur atom, an oxygen atom, a group comprising a nitrogen atom and a hydrogen atom. The introduction of this group(s) can increase the initiation rate of the initiator for the following reasons.

When the group of the initiator comprises the group comprising the nitrogen atom and the hydrogen atom, the group comprising the nitrogen atom and the hydrogen atom can inhibit the quenching of triplet benzophenone by oxygen (O₂). The mechanism is illustrated in FIG. 4. In FIG. 4, R· represents the triplet excited state to which the structural formula (1) transitions after being radiated, and the molecule comprising the nitrogen atom and the hydrogen atom is taken

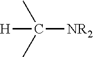

as an example. As illustrated in FIG. 4, the triplet R· has a long lifetime triplet state, which is easy to take away the hydrogen atom from

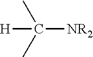

to form RH, and meanwhile an active amine radical

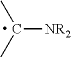

is generated. The generated amine radical

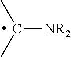

can react with O₂ to generate a peroxide

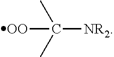

The peroxide

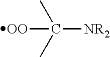

can take away the hydrogen atom of

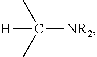

thereby generating another amine radical

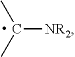

while oxygen does not consume the triplet R· in the whole process. Therefore, when the group of the initiator comprises the group comprising the nitrogen atom and the hydrogen atom, the polymerization inhibition of triplet R· by the oxygen in the curing system can be eliminated. In addition, the generated active amine radical

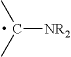

has less steric hindrance and higher initiating activity, which can accelerate the polymerization reaction of carbon-carbon double bonds of ligands on the surface of the quantum dots.

It should be noted that in embodiments of the present disclosure, the group comprising a nitrogen atom and a hydrogen atom may be any appropriate electron-donating group to provide the hydrogen atom. In some embodiments, the group comprising a nitrogen atom and a hydrogen atom may be a pyrrole group or a tertiary amine group. Pyrrole or tertiary amine is more likely to donate hydrogen atoms, turning the group into a more active amine radical, which promotes the polymerization reaction of carbon-carbon double bonds of ligands on the surface of the quantum dots. In some embodiments, the tertiary amine group may be a triethylamine group. In some embodiments, the number of hydrogen atoms may be 1 to 3 in the group comprising a nitrogen atom and a hydrogen atom.

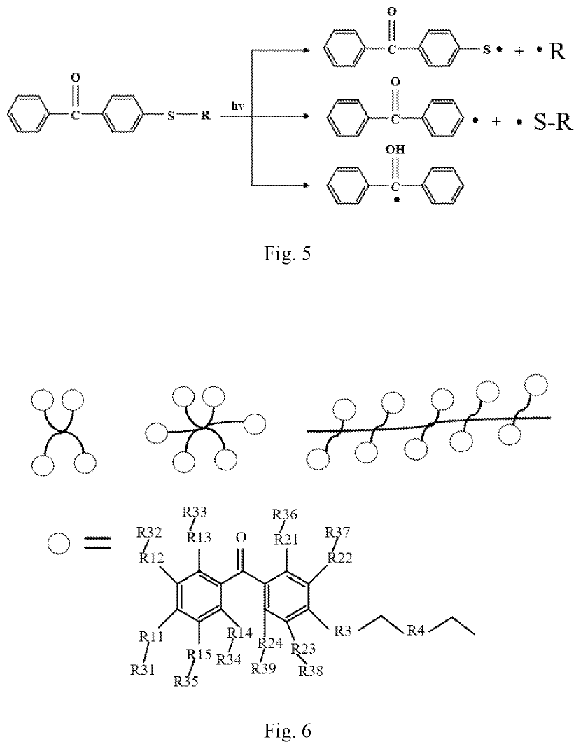

When the group of the initiator comprises a sulfur atom or an oxygen atom, it is also beneficial to increasing the initiation rate of the initiator. Take the sulfur atom as an example, the sulfur atom has an electron-rich effect, which is beneficial to improving the photosensitivity of the initiator. The reaction mechanism is illustrated in FIG. 5. The thioether bond is broken under light radiation of the sulfur atom, generating the sulfur free radical, benzene free radical, etc., which can increase the active centers of free radical and increase the initiation rate of the initiator. The sulfur atom is a weak electron-donating group, which enables the overall structural formula (1) to appear as a push-pull-push conjugated system, causing the absorption of the initiator molecule to be significantly red-shifted. The oxygen atom is also a weak electron-donating group and has a similar effect to the sulfur atom, when the group of the initiator comprises the oxygen atom, the initiation rate of the initiator can also be increased.

In summary, when the group of the structural formula (1) comprises at least one of a sulfur atom, an oxygen atom, and a group comprising a nitrogen atom and a hydrogen atom, the initiation rate of the initiator can be increased. Correspondingly, when the group of the structural formula (1) comprises at least two of a sulfur atom, an oxygen atom, and a group comprising a nitrogen atom and a hydrogen atom, the activity and stability of the free radicals can be greatly improved, thus greatly increases the initiation rate of the initiator, thereby increasing the degree of reaction between the quantum dots and the initiator in the exposure region per unit time, which helps to form a patterned quantum dot light-emitting layer with high quality.

As mentioned before, in the structural formula (1), n is a positive integer greater than or equal to 1. In some embodiments, n is equal to 1. In this case, the structural formula (1) is equivalent to optimizing the molecular structure of a single benzophenone, thereby increasing the initiation rate of the initiator. In alternative embodiments, n may be a positive integer greater than 1, in this case, the structural formula (1) is equivalent to a molecule synthesized with a plurality of optimized benzophenone structural units. In this way, under exposure per unit time, a plurality of initiating sites can be generated simultaneously on one initiator molecule, which can further increase the initiating rate of the initiator per unit time. However, it should be pointed out that theoretically, although the larger the value of n is, the more reaction sites the initiator has, and the more conducive it is to increasing the initiation rate of the initiator, but at the same time, the larger the value of n is, the greater the molecular steric hindrance of the initiator is, and the problem of difficulty in synthesizing and purifying the initiator will occur. Therefore, the value of n is not too large. In some embodiments, the value of n may be a positive integer greater than or equal to 1 and less than or equal to 10. In some embodiments, the value of n may be a positive integer greater than or equal to 1 and less than or equal to 6.

FIG. 6 illustrates the case where n is greater than 1 as an example. For example, the initiator may comprise four, six, or ten optimized benzophenone structural units. In some embodiments, the initiator may also be designed in the form of a polymer.

As mentioned before, the groups R31˜R39 in the structural formula (1) may be present or absent. Since the type of groups R31˜R39 is the same as the type of group R4, when at least one of the groups R31˜R35 is present, it can be understood that the group R4 may also be connected to at least one of the groups R11˜R15; when at least one of the groups R36˜R39 is present, it can be understood that the group R4 may also be connected to at least one of the groups R21˜R24.

In some embodiments, the groups R31˜R39 in the structural formula (1) are absent. In this case, the structural formula (1) can be simplified to be

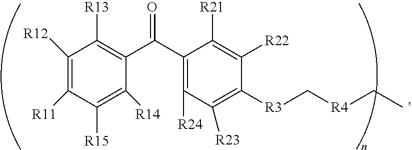

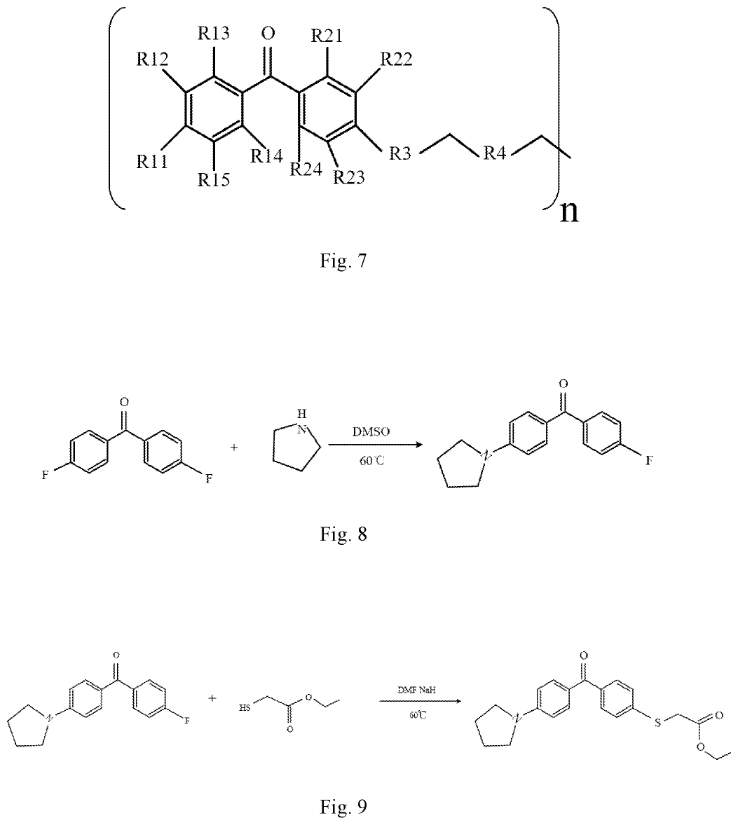

which is hereinafter abbreviated as a structural formula (2), as illustrated in FIG. 7. In the structural formula (2), R11, R12, R13, R14, and R15 may be respectively selected from a hydrogen atom or a group comprising a nitrogen atom and a hydrogen atom, R21, R22, R23 and R24 may be respectively selected from a hydrogen atom or a fluorine atom, R3 may be selected from any one of a sulfur atom, an oxygen atom, an ester group, an amide group and an alkyl group, R4 may be selected from any one of an ester group, an alkyl group, and a group comprising a nitrogen atom and a hydrogen atom, n is a positive integer greater than or equal to 1. Likewise, the group of the structural formula (2) must comprise at least one of the sulfur atom, the oxygen atom and the group comprising the nitrogen atom and the hydrogen atom.

In some embodiments, n in the structural formula (2) is equal to 1, R11 is a pyrrole group, R12, R13, R14, and R15 are hydrogen atoms, R21˜R24 are hydrogen atoms, R3 is a sulfur atom, and R4 is an ester group, that is, the structural formula (2) is specifically

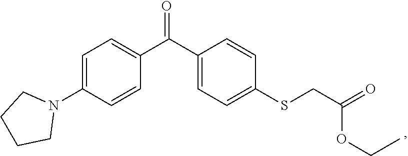 [SMILES: CCOC(=O)CSc1ccc(C(=O)c2ccc(N3CCCC3)cc2)cc1]

which is abbreviated as SBP.

The synthesis steps of the initiator SBP are described below with an example.

As illustrated in FIG. 8, mix a certain amount of 4,4-difluorobenzophenone (for example, about 218.2 mg) and tetrahydropyrrole (for example, about 71.2 mg), add an appropriate amount of dimethyl sulfoxide DMSO (for example, about 6 mL), and react at 60° C. for about 6 hours. Then, the reacted solution is poured into 100 mL of water and filtered to obtain a solid powder. The solid powder is washed with methanol to remove the raw material 4,4-difluorobenzophenone in the solid powder to obtain an intermediate product

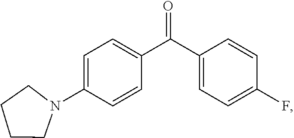 [SMILES: O=C(c1ccc(F)cc1)c1ccc(N2CCCC2)cc1]

which is abbreviated as NBP—F.

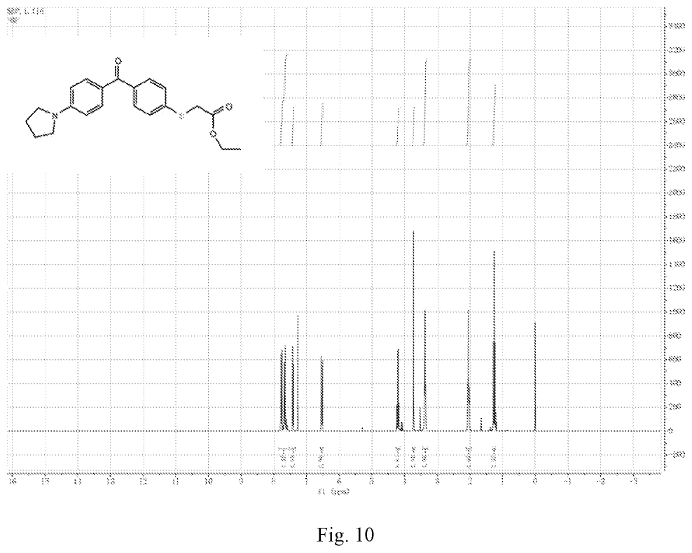

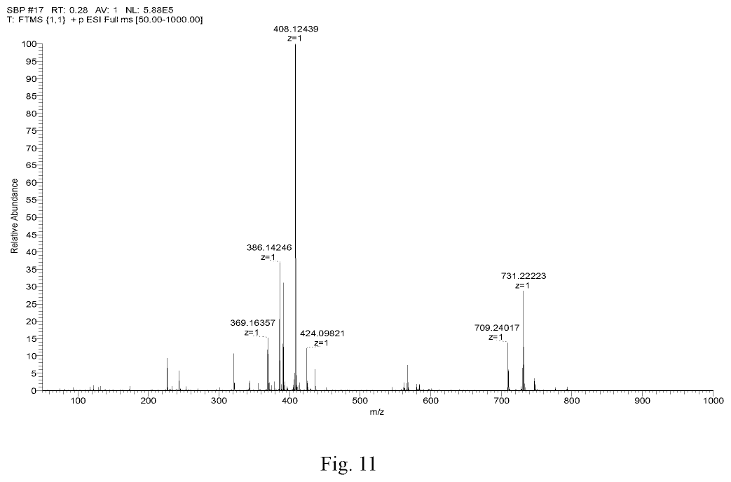

As illustrated in FIG. 9, mix a certain amount of NBP—F (for example, about 270 mg) and ethyl thioglycolate (for example, about 120 mg), add an appropriate amount of NaH (for example, about 24 mg) and anhydrous DMF (for example, about 10 mL), and heat at 60° C. for 8 hours. Then, the reacted solution is poured into 100 mL of water and filtered to obtain solid powder. The solid powder is purified by column chromatography to obtain the target product SBP. The developing agents used in column chromatography are methylene chloride and ethyl acetate, and the ratio of the two may be 10:1.

FIG. 10 illustrates the hydrogen nuclear magnetic resonance spectrum of SBP, and FIG. 11 illustrates the mass spectrum of SBP. The hydrogen nuclear magnetic resonance spectrum and mass spectrum show that the synthetic structure of SBP is correct.

The synthesis steps of SBP are relatively simple and are easy to implement in terms of process. In the molecular structural formula of SBP, the group R11 is selected as a pyrrole group. As mentioned before, the pyrrole group is an electron-donating group and is more likely to provide hydrogen atoms. Under light radiation, the pyrrole group can eliminate the polymerization inhibition of triplet benzophenone structural unit by oxygen in the curing system, and the generated active amine radical has less steric hindrance and higher initiating activity, which can accelerate the polymerization reaction of carbon-carbon double bonds of ligands on the surface of the quantum dots. The group R3 is selected as an S atom. As mentioned above, the S atom can generate S free radical under light radiation, increasing the concentration and type of free radicals per unit time, thereby accelerating the polymerization rate of free radicals per unit time. The introduction of both pyrrole group and S atom into the initiator SBP can greatly increase the initiation rate of the initiator SBP. In addition, the group R3 is para-position with C═O, which makes the synthesis of SBP easier to achieve. The group R4 is selected as an ester group, which can improve the compatibility between the initiator SBP and the quantum dot solvent, making the material system more stable.

In some embodiments, it can be further synthesized on the basis of SBP to obtain

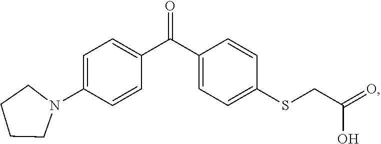 [SMILES: O=C(O)CSc1ccc(C(=O)c2ccc(N3CCCC3)cc2)cc1]

which is abbreviated as SBP—COOH. The synthesis steps of SBP—COOH are as follows: mix a certain amount of SBP (for example, about 408 mg) and NaOH (for example, about 0.04 mg), add an appropriate amount of acetone (for example, about 10 mL), and react at 40° C. for 4 hours. Then, the acetone is evaporated to dryness to obtain a yellow solid. The yellow solid is washed three times with water to remove NaOH. Then, it is drained to obtain the target product SBP—COOH.

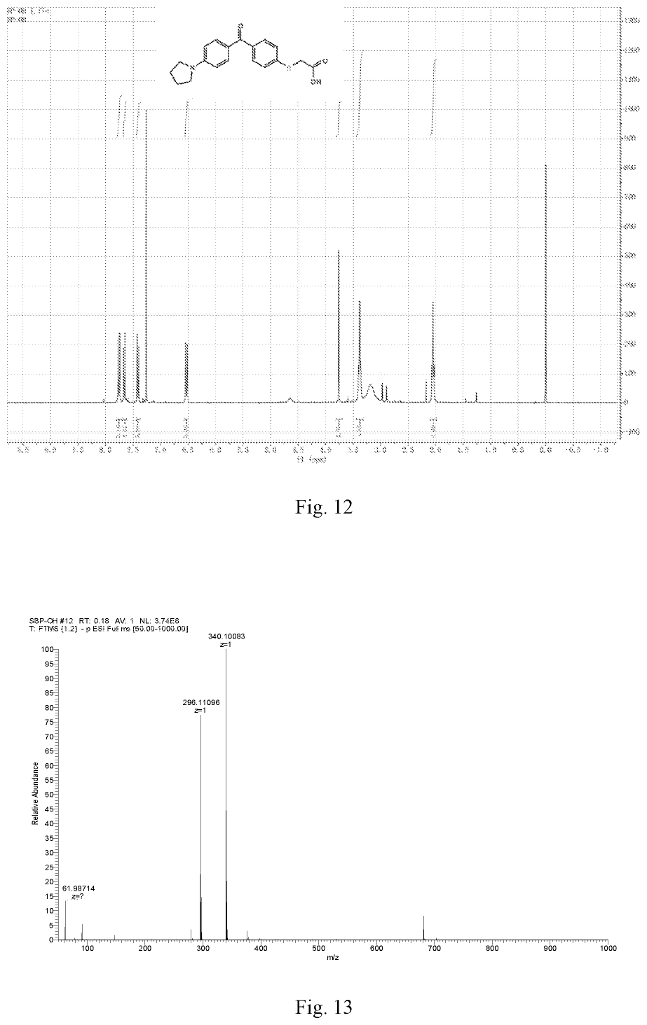

FIG. 12 illustrates the hydrogen nuclear magnetic resonance spectrum of SBP—COOH, and FIG. 13 illustrates the mass spectrum of SBP—COOH. The hydrogen nuclear magnetic resonance spectrum and mass spectrum show that the synthetic structure of SBP—COOH is correct.

In alternative embodiments, n in the structural formula (2) is equal to 2, R11 is a pyrrole group, R12, R13, R14, and R15 are hydrogen atoms, R21˜R24 are hydrogen atoms, R3 is a sulfur atom, and R4 is an ester group, that is, the structural formula (2) is specifically

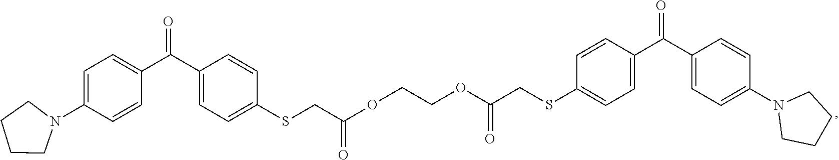 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]

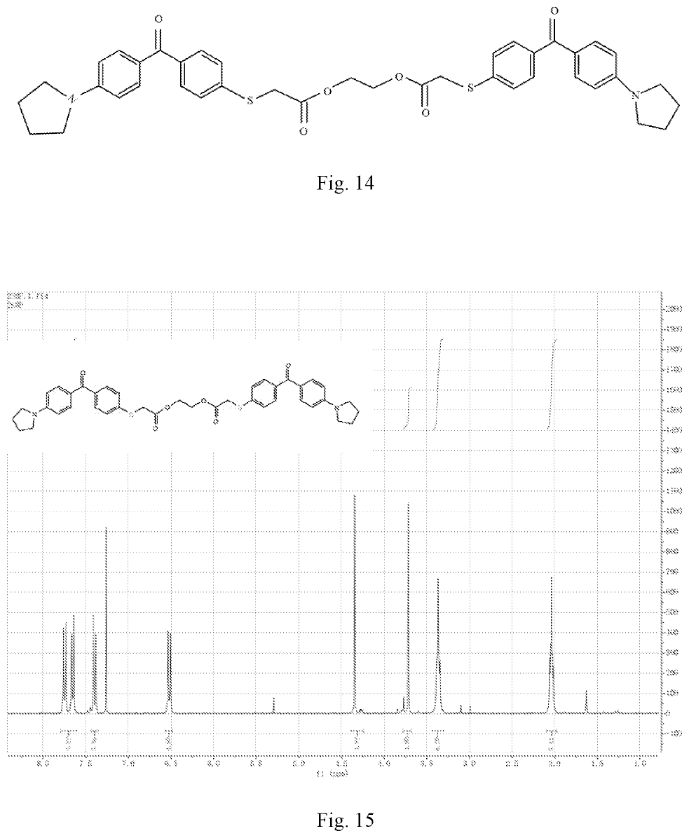

which is abbreviated as (SBP)₂, as illustrated in FIG. 14.

The synthesis steps of the initiator (SBP)₂ are described below as an example.

Mix and dissolve a certain amount of SBP—COOH (for example, about 340 mg), ethylene glycol (for example, about 62 mg), 1-ethyl-(3-dimethylaminopropyl) carbamide diimide (EDC for short, for example, about 150 mg), 4-dimethylaminopyridine (DMAP for short, for example, about 50 mg), and dichloromethane (for example, about 10 mL) and react at room temperature for 12 hours. Purify using column chromatography to obtain the target product (SBP)₂. The developing agent used in column chromatography is methylene chloride.

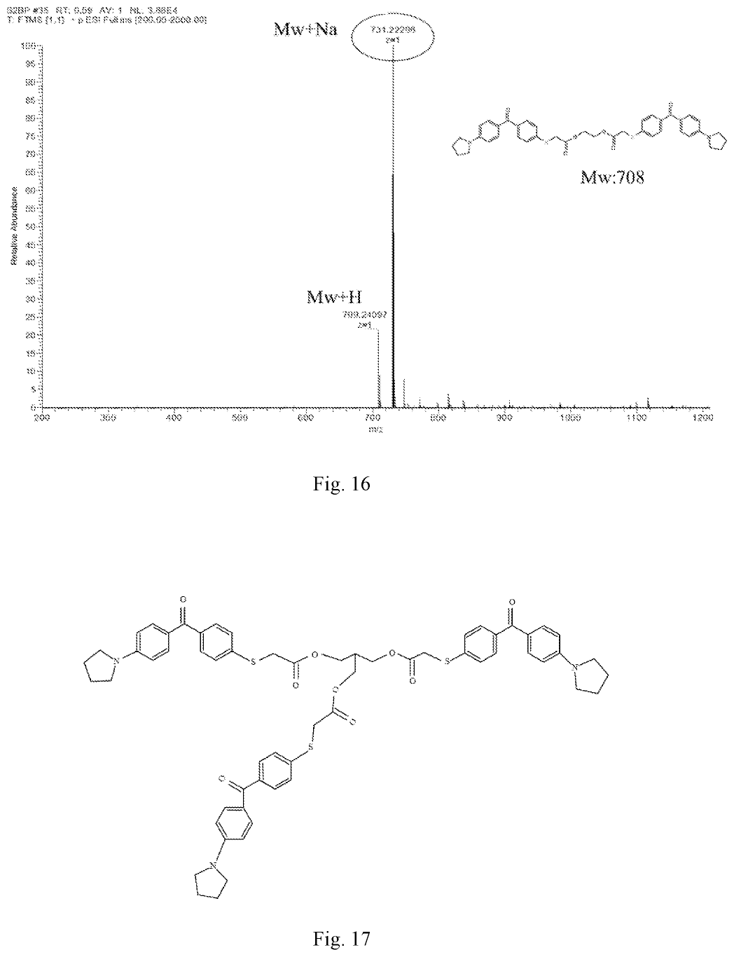

FIG. 15 illustrates the hydrogen nuclear magnetic resonance spectrum of (SBP)₂, and FIG. 16 illustrates the mass spectrum of (SBP)₂. The relative molecular mass of (SBP)₂ is 708. FIG. 16 illustrates two peaks, respectively are 731 and 709. These two peaks correspond to the relative molecular mass of (SBP) 2+Na (731) and the relative molecular mass of (SBP)₂+H (709), respectively, which indicates that the synthetic structure of (SBP)₂ is correct.

In addition to the technical effects of the initiator SBP, the initiator (SBP)₂ also has more initiating sites than the initiator SBP. Therefore, the initiation rate of the initiator (SBP)₂ per unit time is higher, which helps to further enhance the cross-link effect of quantum dots.

In alternative embodiments, n in the structural formula (2) is equal to 3, R11 is a pyrrole group, R12, R13, R14, and R15 are hydrogen atoms, R21˜R24 are hydrogen atoms, R3 is a sulfur atom, and R4 is an ester group. That is, the structural formula (2) is specifically

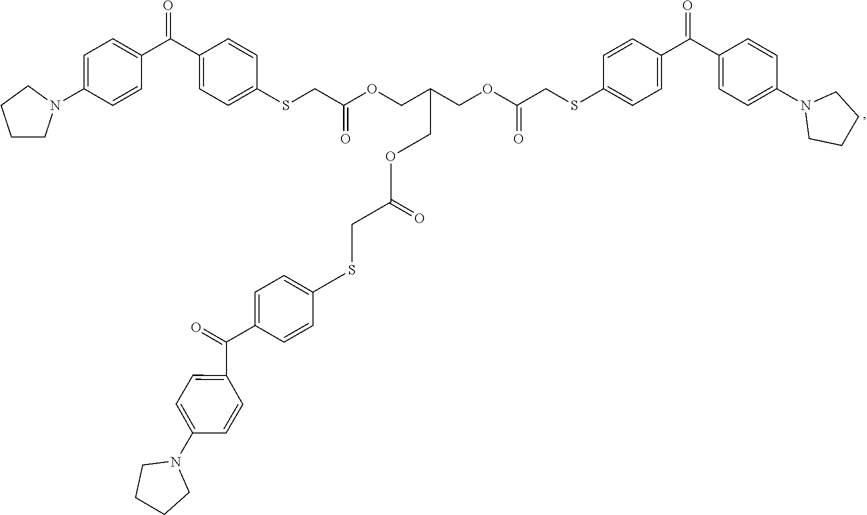 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(=O)c2ccc(N3CCCC3)cc2)cc1]

which is abbreviated as (SBP)₃, and the structural formula of (SBP)₃ is illustrated in FIG. 17.

The synthesis steps of the initiator (SBP)₃ are described below as an example.

Mix and dissolve a certain amount of SBP—COOH (for example, about 510 mg), glycerol (for example, about 62 mg), EDC (for example, about 200 mg), DMAP (for example, about 70 mg), and dichloromethane (for example, about 10 mL), and react at room temperature for 12 hours. Purify using column chromatography to obtain the target product (SBP)₃. The developing agent used in column chromatography is dichloromethane.

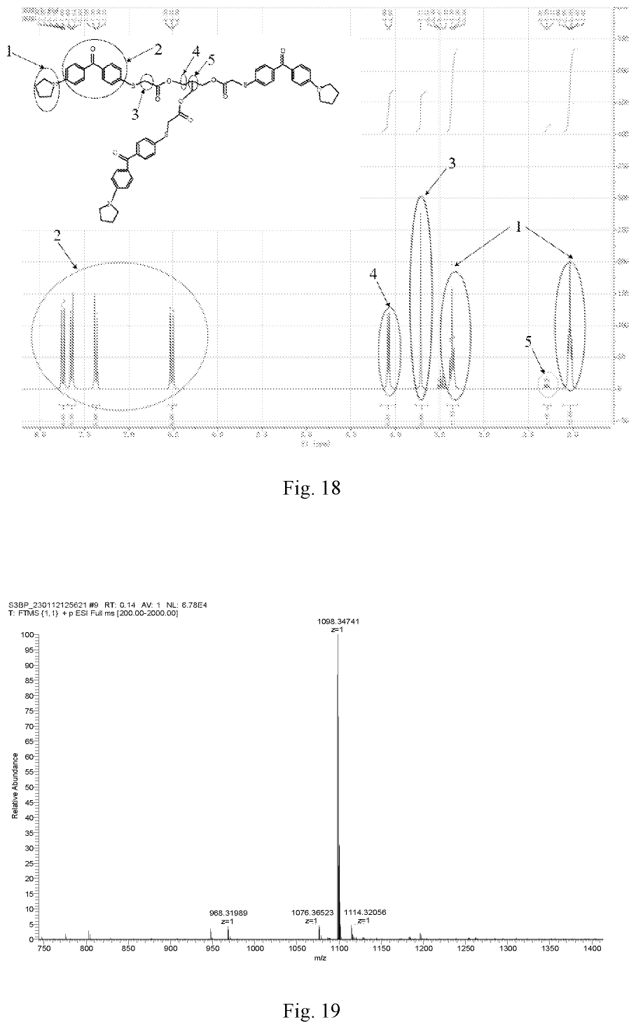

FIG. 18 illustrates the hydrogen nuclear magnetic resonance spectrum of (SBP)₃, and FIG. 19 illustrates the mass spectrum of (SBP)₃. In FIG. 18, the two peaks labeled “1” represent the group labeled “1” in the molecular structure (SBP)₃, the peak labeled “2” represents the group labeled “2” in the molecular structure (SBP)₃, the peak labeled “3” represents the group labeled “3” in the molecular structure (SBP)₃, the peak labeled “4” represents the group labeled “4” in the molecular structure (SBP)₃, and the peak labeled “5” represents the group labeled “5” in the molecular structure (SBP)₃. The relative molecular mass of (SBP)₃ is 1098, and the value of the peak illustrated in FIG. 19 is 1098. FIGS. 18 and 19 indicate that the synthesized structure of (SBP)₃ is correct.

In addition to the technical effects of the initiator SBP, the initiator (SBP)₃ has more initiating sites than initiators SBP and (SBP)₂. Therefore, the initiator (SBP)₃ has higher initiation rate per unit time, resulting in better cross-link of quantum dots.

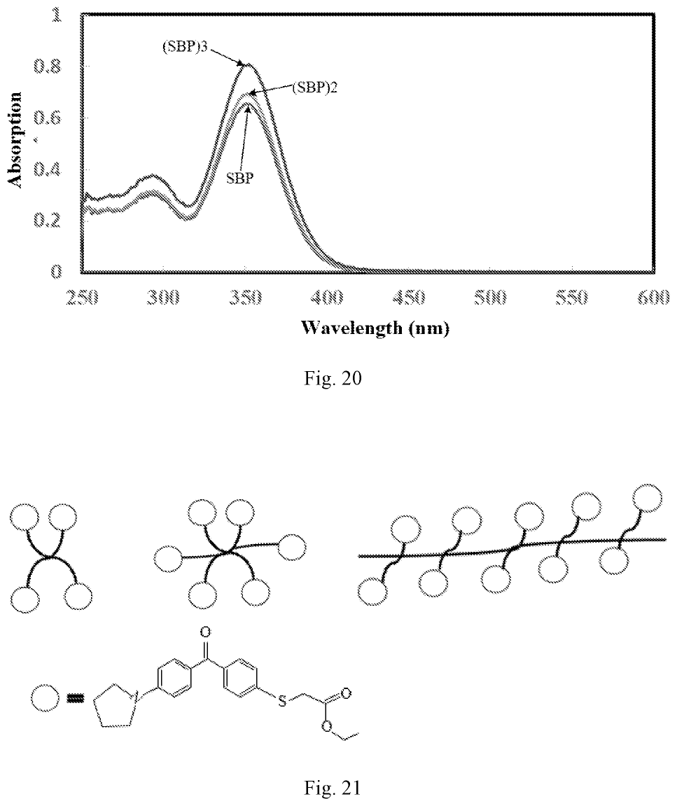

FIG. 20 illustrates the ultraviolet absorption spectra of three initiators SBP, (SBP)₂, and (SBP)₃. As illustrated in FIG. 20, at the same solubility (0.01 mg/mL), three initiators have strong absorption at 365 nm, wherein (SBP)₃ has the strongest absorption intensity, (SBP)₂ has the middle absorption intensity, and SBP has the weakest absorption intensity.

In the initiators SBP, (SBP)₂, and (SBP)₃, both the pyrrole group and the sulfur atom are introduced. In this way, on the one hand, under light radiation, the initiator can capture a hydrogen atom from the pyrrole group, turning the pyrrole group into an active amine free radical. The generated amine free radical can then react with oxygen to generate peroxide. The peroxide can capture a hydrogen atom, thereby generating another amine radical, while oxygen does not consume the triplet benzophenone structural unit in the entire process. Therefore, the introduction of pyrrole group can eliminate the polymerization inhibition of free radical polymerization reaction by oxygen in the system. Moreover, the active amine free radical has little steric hindrance and high initiating activity, which can accelerate the polymerization reaction of carbon-carbon double bonds of ligands on the surface of the quantum dots. On the other hand, the thioether bond is broken under light radiation of the sulfur atom, generating the sulfur radical, benzene radical, etc., which increases the active centers of free radical. The sulfur atom is a weak electron-donating group, which enables the overall structural formula of the initiator to appear as a push-pull-push conjugated system, causing the absorption of the entire molecule to be significantly red-shifted. Therefore, by simultaneously introducing the pyrrole group and the sulfur atom into the molecular structure, the activity and stability of free radicals can be greatly improved, thereby greatly increasing the initiation rate of the initiator. In turn, the degree of reaction between quantum dots and initiator in the exposed region can be increased per unit time, which helps to form a patterned quantum dot light-emitting layer with high quality. The group R4 is selected as an ester group, which can improve the compatibility between the initiator SBP and the quantum dot solvent, making the material system more stable.

In some embodiments, n in the structural formula (2) is equal to 4, R11 is a pyrrole group, R12, R13, R14, and R15 are hydrogen atoms, R21˜R24 are hydrogen atoms, R3 is a sulfur atom, and R4 is an ester group, that is, the structural formula (2) is specifically

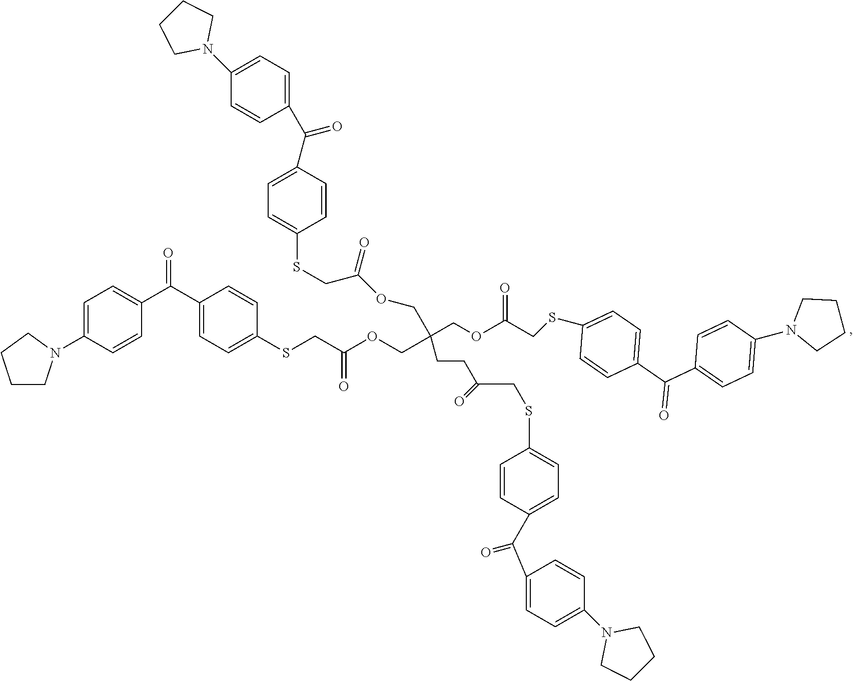 [SMILES: O=C(CCC(COC(=O)CSc1ccc(C(=O)c2ccc(N3CCCC3)cc2)cc1)(COC(=O)CSc1ccc(C(=O)c2ccc(N3CCCC3)cc2)cc1)COC(=O)CSc1ccc(C(=O)c2ccc(N3CCCC3)cc2)cc1)CSc1ccc(C(=O)c2ccc(N3CCCC3)cc2)cc1]

which is abbreviated as (SBP)₄.

In some embodiments, n in the structural formula (2) may also be a larger positive integer, for example, n may be 5, 6, 10 or a larger positive integer, as illustrated in FIG. 21. In this way, introducing multiple benzophenone units on the basis of optimized benzophenone molecule can increase the initiating sites and make the initiator have higher initiating rate per unit time. It can ensure that the initiator has a higher degree of photopolymerization under the same exposure dose, which can further improve the photo-patterning effect of the initiator and promote the cross-link effect of quantum dots.

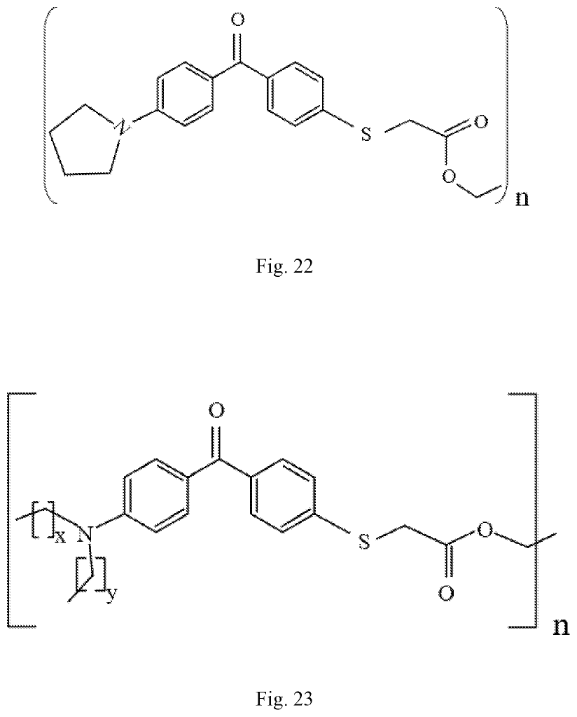

FIG. 22 illustrates the structural formula

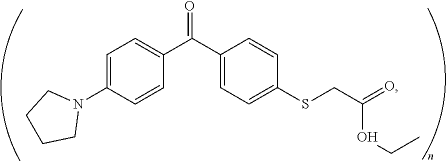

of the initiator, where n is a positive integer greater than or equal to 1. When n is equal to 1, it is SBP; when n is equal to 2, it is (SBP)₂; when n is equal to 3, it is (SBP)₃.

In the above embodiments, the case where the group comprising the nitrogen atom and the hydrogen atom is a pyrrole group is described. In alternative embodiments, the group comprising the nitrogen atom and the hydrogen atom may also be a tertiary amine group.

FIG. 23 illustrates the structural formula

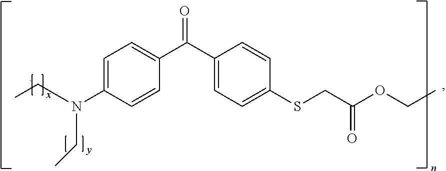

which is abbreviated as a structural formula (3). That is, when R11 in the structural formula (2) is a tertiary amine group, R12, R13, R14, and R15 are hydrogen atoms, R21˜R24 are hydrogen atoms, R3 is a sulfur atom, and R4 is an ester group, the structural formula (2) is embodied as the structural formula (3). In the structural formula (3), both x and y are positive integers greater than or equal to 1. Since both the tertiary amine group and the pyrrole group can easily provide hydrogen atoms, the initiator

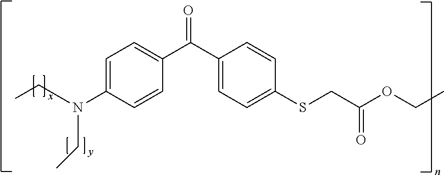

has similar technical effects to the initiator

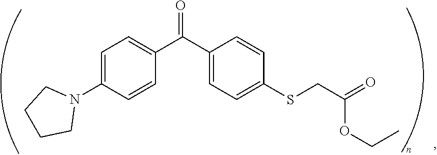

For the sake of simplicity, the technical effects of

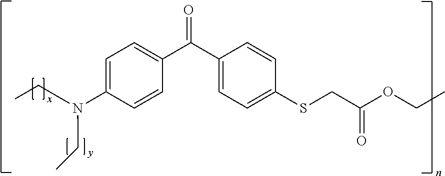

will not be repeated.

In some embodiments, in the structural formula (3), the number of carbon atoms in the branch chain connected to the N atom may be 2˜30, such as 2, 3, 6, 9, 16, 20, 30, etc.

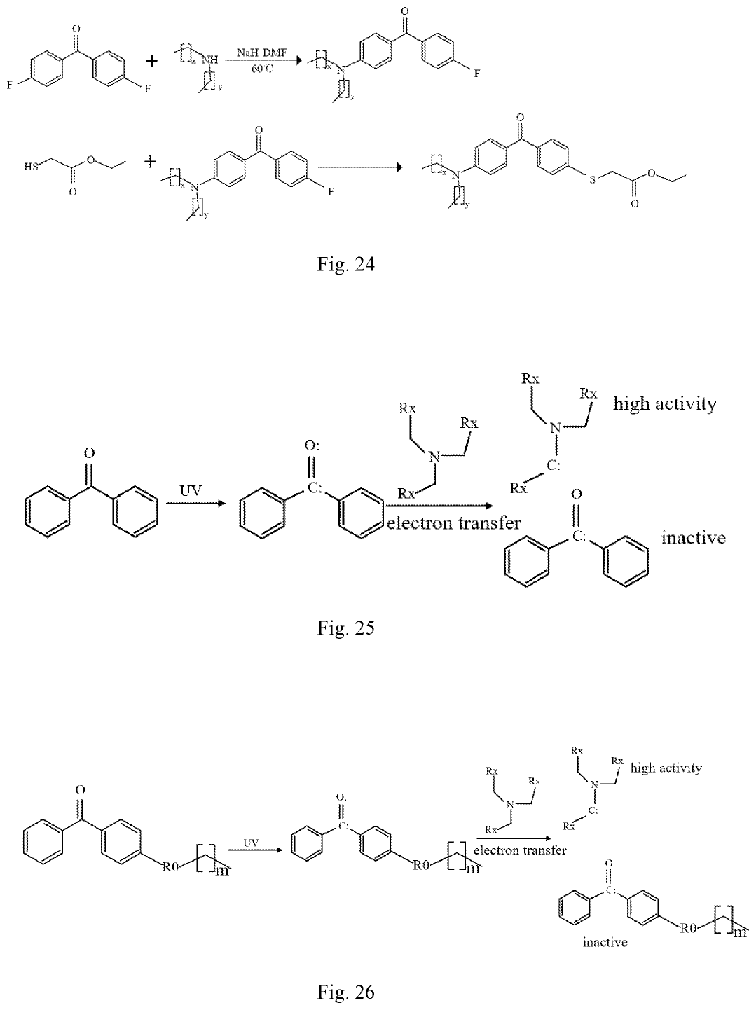

FIG. 24 illustrates the synthesis steps of the initiator

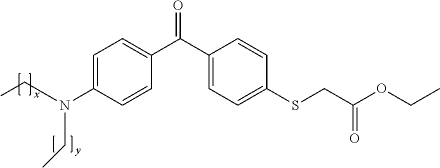

(i.e. when n=1 in the structural formula (3)). As illustrated in FIG. 24, mix the 4,4-difluorobenzophenone and the tertiary amine

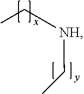

add an appropriate amount of NaH and anhydrous DMF, and react at 60° C. for a period of time, to obtain an intermediate product

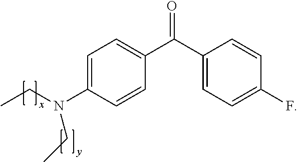

This intermediate product is then mixed and reacted with ethyl thioglycolate to generate the product

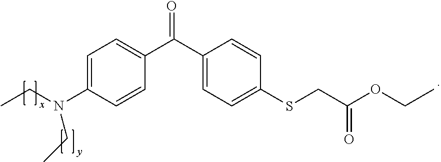

The synthesis steps of

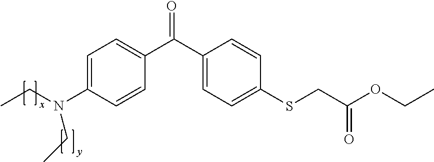

are relatively simple and easy to implement in terms of process. In this molecular structural formula, the group R11 is selected as a tertiary amine group. As mentioned before, the tertiary amine group is an electron-donating group and is more likely to provide hydrogen atoms. Under light radiation, it can eliminate the polymerization inhibition of triplet benzophenone structural unit by oxygen in the curing system. Moreover, the generated active amine radical has less steric hindrance and higher initiating activity, which can accelerate the polymerization reaction of carbon-carbon double bonds of ligands on the surface of the quantum dots. The group R3 is selected as an S atom. As mentioned above, the S atom can generate S free radical under light radiation, increasing the concentration and type of free radicals per unit time, thereby accelerating the polymerization rate of free radicals per unit time. Introducing both the tertiary amine group and the S atom into the initiator can greatly increase the initiation rate of the initiator. In addition, S atom is para-position with C═O, which makes the synthesis of this initiator easier to achieve. The group R4 is selected as an ester group, which can improve the compatibility between the initiator and the quantum dot solvent, making the material system more stable.

According to another aspect of the present disclosure, another initiator is provided, the initiator is a mixture of a benzophenone and an amine compound or a mixture of a benzophenone derivative and an amine compound. This initiator has similar technical effects to the initiator described in the previous embodiments and can also increase the initiation rate.

In some embodiments, the amine compound may be a tertiary amine or pyrrole. It is easier for benzophenone or benzophenone derivative to capture hydrogen atoms from the tertiary amine or pyrrole, which allows the tertiary amine or pyrrole to form an amine free radical with higher activity. The structural formula of the benzophenone derivative is

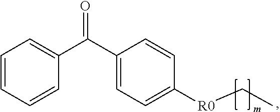

where R0 is selected from any one of O, S, C₆H₅, OH, Br, Cl, and I, and m is a positive integer greater than or equal to 0.

FIG. 25 is a schematic diagram showing the principle that the mixture of benzophenone and amine compound can improve the initiation rate of initiator, FIG. 26 is a schematic diagram showing the principle that the mixture of benzophenone derivative and amine compound can improve the initiation rate of initiator, wherein the amine compound is taken the tertiary amine

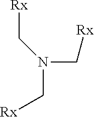

as an example, but this is only an example of the amine compound and does not limit the type of the amine compound. As mentioned before, the benzophenone will transition to a triplet benzophenone under light radiation, but the triplet benzophenone is easily quenched by O₂ in the air, weakening its polymerization activity. However, in embodiments of the present disclosure, as illustrated in FIGS. 25 and 26, an improved initiator is provided by mixing the benzophenone with the amine compound

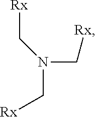

or by mixing the benzophenone derivative with the amine compound

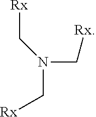

Under light radiation, electron transfer occurs between the benzophenone and the amine compound to generate a highly active amine free radical

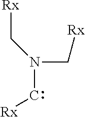

and an inactive

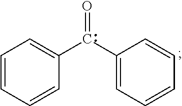 [SMILES: O=C(c1ccccc1)c1ccccc1]

alternatively, electron transfer occurs between the benzophenone derivative and the amine compound to generate a highly active amine free radical

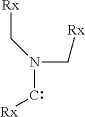

and an inactive

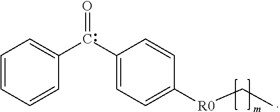

The highly active amine free radical will not be quenched by O₂ in the air and can maintain relatively high activity in O₂, thereby initiating the polymerization of the carbon-carbon double bonds of ligands of the quantum dots. Therefore, the initiator formed by mixing the amine compound with the benzophenone or the benzophenone derivative can eliminate the polymerization inhibition of polymerization reaction of free radicals by oxygen in the curing system, which is beneficial to promoting the polymerization of ligands of quantum dots and forming a patterned quantum dot light-emitting layer with high quality.

In some embodiments, the group Rx in the tertiary amine

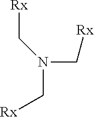

comprises, but is not limited to, H, CH₃, CH₂CH₃, etc. For example,

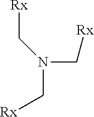

may be triethylamine.

In some embodiments, the molar ratio of benzophenone or benzophenone derivative to the tertiary amine in the mixture may be 1:1. After light radiation, the benzophenone or benzophenone derivative in the mixture captures a hydrogen atom from the tertiary amine. One benzophenone molecule or one benzophenone derivative molecule only needs one tertiary amine molecule, the electron transfer requirement between the benzophenone molecule or benzophenone derivative molecule and the tertiary amine molecule can be met.

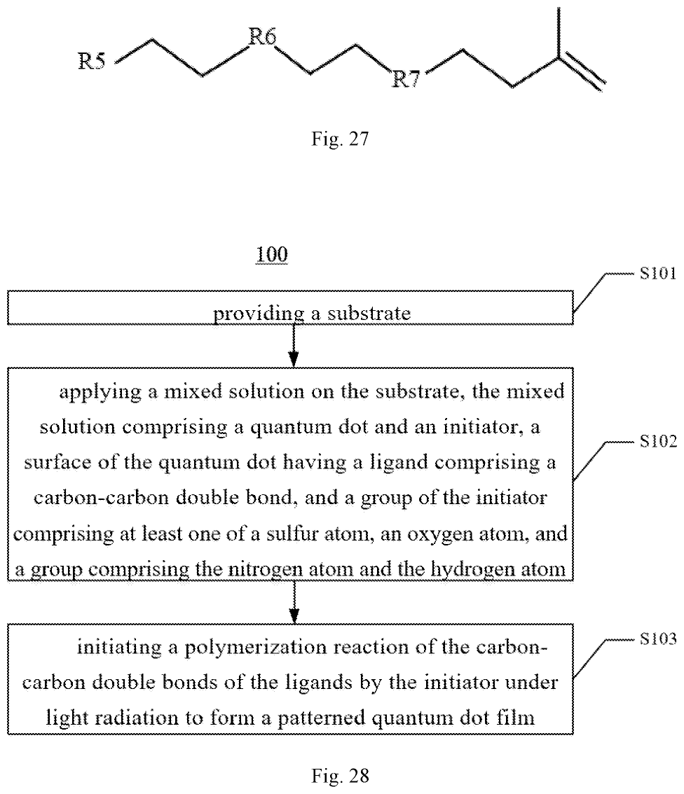

According to yet another aspect of the present disclosure, a quantum dot is provided, and the surface of the quantum dot has a ligand. FIG. 27 illustrates the structural formula

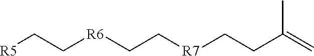

of the ligand on the surface of the quantum dot, which is hereinafter abbreviated as a structural formula (4). In the structural formula (4), R5 is coordinately connected to the surface of the quantum dot and is selected from any one of a sulfhydryl group, a carboxyl group and an amino group, R6 is selected from an ester group or an ether group, and R7 is selected from an ester group or an ether group. The ligands on the surface of the quantum dots can react chemically with the initiators described in any of the previous embodiments under light radiation.

The organic ligands on the surface of quantum dots can fill defects on the surface of quantum dots, thereby improving the stability and quantum yield of quantum dots. The initiator provided in the embodiments of the present disclosure is a free radical photo-initiator, which can initiate the polymerization reaction of carbon-carbon double bonds. In order to achieve the photo-patterning effect of quantum dots, the ligand on the surface of quantum dots needs to have the carbon-carbon double bond. In some embodiments, the number of carbon-carbon double bond in the ligand is 1. In some embodiments, the number of carbon-carbon double bonds in the ligand may be 2˜4 to increase the degree of cross-link. However, when the number of carbon-carbon double bonds in the ligand is too large, it may be not beneficial to the colloidal stability of the quantum dots themselves.

In addition, the group R5 in the ligand needs to coordinate with the defect state of the quantum dot. Therefore, R5 needs to comprise functional groups such as the sulfhydryl group, the carboxyl group or the amino group. In addition, considering that the ligand of quantum dot comprises the carbon-carbon double bond, in order to improve the solvent compatibility between the initiator and the quantum dot, the ligand of quantum dot also needs to comprise functional groups such as the ester group or the ether group, namely the groups R6 and R7, which can improve the mutual solubility of quantum dots and initiator in the solvent.

In some embodiments, the group R5 of the ligand of the quantum dot is a carboxyl group, and the groups R6 and R7 are respectively an ester group. In this case, the structural formula (4) of the ligand of the quantum dot becomes

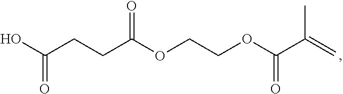 [SMILES: C=C(C)C(=O)OCCOC(=O)CCC(=O)O]

which is abbreviated as MMES. Quantum dots with the ligand MMES have good dispersion in the propylene glycol methyl ether acetate (PGMEA for short) solvent, for example, the solubility may be greater than 60 mg/mL.

In the structural formula (4) of the ligand of the quantum dot, the carbon-carbon double bond is designed to be located at the end of the molecular chain, which is beneficial to reducing the steric hindrance of free radical polymerization and improving the activity of free radical polymerization.

In some embodiments, the number of carbon atoms in the structural formula (4) of the ligand of the quantum dot is 2˜30, for example, the number of carbon atoms is 2, 5, 10, 16, 20, 25, 30, etc.

The quantum dots provided by the embodiments of the present disclosure may be any appropriate quantum dots, comprising but not limited to any one of IIB-VIA quantum dots, IIIA-VA quantum dots, IVA-VIA quantum dots, quantum dots with the core-shell structure, perovskite quantum dots, nanorods, nanosheets, and cadmium (Cd)-free quantum dots.

FIG. 28 illustrates a flow chart of a method 100 of preparing a patterned quantum dot light-emitting layer. As illustrated in FIG. 28, the method 100 comprises the following steps:

S101: providing a substrate.

The substrate may be an inorganic material, an organic material, a silicon wafer, a composite material layer, etc. Examples of inorganic material may be glass, metal, etc., examples of organic material may be polycarbonate, polymethyl methacrylate, polyethylene terephthalate, polyethylene naphthalate, polyamide, polyethersulfone, or combinations thereof.

S102: applying a mixed solution on the substrate, the mixed solution comprising the quantum dot and the initiator, a surface of the quantum dot having the ligand comprising the carbon-carbon double bond, and the group of the initiator comprising at least one of the sulfur atom, the oxygen atom, and the group comprising the nitrogen atom and the hydrogen atom.

The quantum dot may be the quantum dot described in any of the previous embodiments, and the initiator may be the initiator described in any of the previous embodiments.

In some embodiments, the concentration of quantum dots in the mixed solution may be about 25˜30 mg/mL, such as 25 mg/mL, 27.5 mg/mL, 30 mg/mL, and the like. The concentration of the initiator in the mixed solution may be about 0.1˜1.0 mg/mL, such as 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, and the like.

S103: initiating a polymerization reaction of the carbon-carbon double bonds of the ligands by the initiator under light radiation to form the patterned quantum dot light-emitting layer.

In the method 100, a photosensitive reaction occurs between the initiator and the ligand of the quantum dot to form the patterned quantum dot light-emitting layer. The reactivity between the initiator and the ligand of the quantum dot determines the stability of the patterned quantum dot light-emitting layer. In embodiments of the present disclosure, the group of the initiator comprises at least one of the sulfur atom, the oxygen atom, and the group comprising the nitrogen atom and the hydrogen atom by designing and optimizing the molecule of the initiator, which can increase the initiation rate per unit time and make the activity of the free radical more stable, thereby increasing the degree of photoreaction between the quantum dots and the initiator in the exposed region and reducing the exposure dose, thus helping to form the patterned quantum dot light-emitting layer with high quality.

The quantum dots provided in the embodiments of the present disclosure are negative glue type quantum dot materials. Specifically, during the preparation process, the mixed solution comprising quantum dots and initiator solidifies into a film. Under light radiation, the chemical composition of the exposed portion of the film changes but the chemical composition of the unexposed portion of the film does not change. The unexposed portion of the film can subsequently be dissolved in a specific developer, while the exposed portion is not dissolved by the developer so as to form the patterned quantum dot light-emitting layer. Therefore, during preparation, the quantum dot film with the photosensitive property can be directly exposed and developed to form the patterned quantum dot light-emitting layer without using photoresist and without etching the quantum dot light-emitting layer.

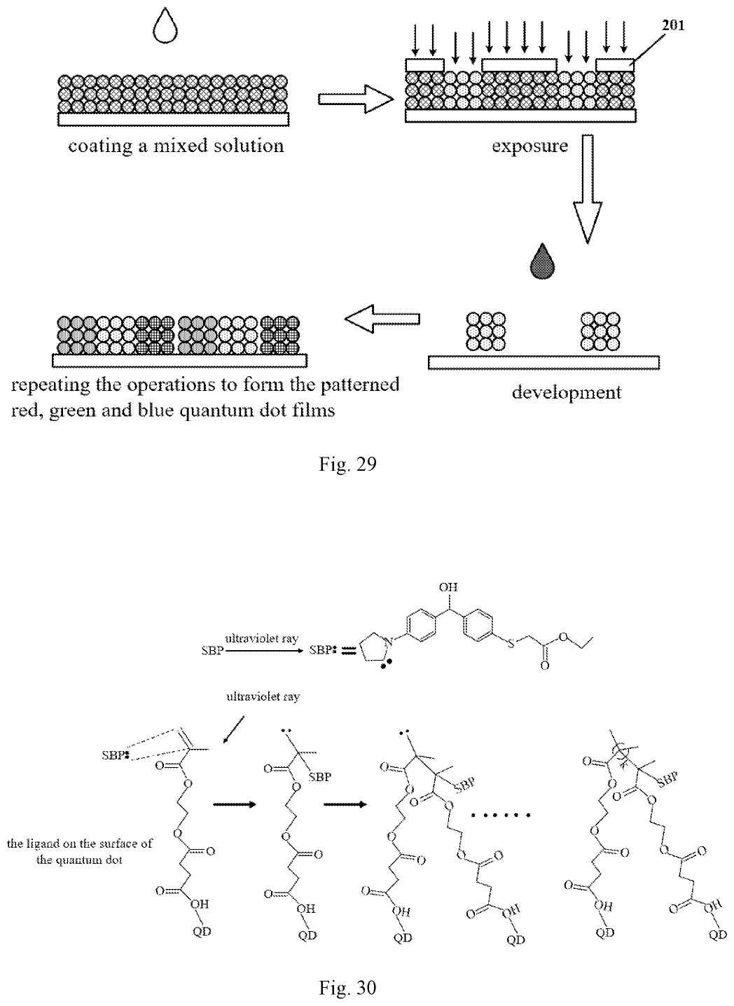

FIG. 29 illustrates a schematic diagram of a method for preparing a patterned quantum dot light-emitting layer. Next, the steps of the method 100 will be described in more detail with reference to FIG. 29.

First, a first mixed solution comprising the first quantum dots and the initiator is coated on the substrate. The first mixed solution applied on the substrate is cured to form a first intermediate layer.

Then, the mask 201 is aligned with the substrate so that the mask 201 exposes the first target region and blocks the non-target region, and the first intermediate layer is radiated with light of a specific wavelength (e.g., ultraviolet light). Under light radiation, the initiator initiates a polymerization reaction of the carbon-carbon double bonds of the ligands on the surface of the quantum dots, so that the chemical composition of the portion of the first intermediate layer exposed by the mask 201 changes under light radiation.

Then, the first intermediate layer after exposure is developed by using a developer, the portion of the first intermediate layer located in the non-target region is dissolved by the developer, and the portion of the first intermediate layer located in the first target region is not dissolved by the developer so as to be left. After development, processes such as thermal baking annealing can be performed to form a patterned first quantum dot light-emitting layer in the first target region. For example, the first quantum dot light-emitting layer may be used to emit red light.

By repeating the above operations, a patterned second quantum dot light-emitting layer and a patterned third quantum dot light-emitting layer can be formed respectively. Specifically, a second mixed solution comprising the second quantum dots and the initiator is coated on the substrate on which the first quantum dot light-emitting layer is formed. The second mixed solution applied on the substrate is cured to form a second intermediate layer. Then, the mask 201 is aligned with the substrate so that the mask 201 exposes the second target region and blocks the non-target region, and the second intermediate layer is radiated with light of a specific wavelength (e.g., ultraviolet light). Under light radiation, the initiator initiates a polymerization reaction of the carbon-carbon double bonds of the ligands on the surface of the quantum dots, so that the chemical composition of the portion of the second intermediate layer exposed by the mask 201 changes under light radiation. Then, the second intermediate layer after exposure is developed by using a developer, the portion of the second intermediate layer located in the non-target region is dissolved by the developer, and the portion of the second intermediate layer located in the second target region is not dissolved by the developer so as to be left. After development, processes such as the thermal baking annealing can be performed to form the patterned second quantum dot light-emitting layer in the second target region. For example, the second quantum dot light-emitting layer may be used to emit green light. Then, a third mixed solution comprising the third quantum dots and the initiator is coated on the substrate on which the first quantum dot light-emitting layer and the second quantum dot light-emitting layer are formed. The third mixed solution applied on the substrate is cured to form a third intermediate layer. Then, the mask 201 is aligned with the substrate so that the mask 201 exposes the third target region and blocks the non-target region, and the third intermediate layer is radiated with light of a specific wavelength (e.g., ultraviolet light). Under light radiation, the initiator initiates a polymerization reaction of the carbon-carbon double bonds of the ligands on the surface of the quantum dots, so that the chemical composition of the portion of the third intermediate layer exposed by the mask 201 changes under light radiation. Then, the third intermediate layer after exposure is developed by using a developer, the portion of the third intermediate layer located in the non-target region is dissolved by the developer, and the portion of the third intermediate layer located in the third target region is not dissolved by the developer so as to be left. After development, processes such as the thermal baking annealing can be performed to form the patterned third quantum dot light-emitting layer in the third target region. For example, the third quantum dot light-emitting layer may be used to emit blue light.

It should be noted that the content of the initiator in the first quantum dot light-emitting layer, the second quantum dot light-emitting layer, and the third quantum dot light-emitting layer may be same or different from each other.

Through the above method, a patterned red, green, and blue quantum dot light-emitting layer can be formed on the substrate. Compared with the traditional method which uses the photoresist to block to pattern the quantum dot light-emitting layer, the quantum dot patterning method provided by the embodiments of the present disclosure does not require photoresist, can directly expose and develop the quantum dot film, the process flow is simple, and there are no problems such as solvent compatibility, so that the patterned quantum dot light-emitting layer has higher quality. Using this patterning method is beneficial to forming a quantum dot light-emitting diode device with higher resolution and higher quality.

In some embodiments, the structural formula of the ligand on the surface of the quantum dot in the mixed solution is

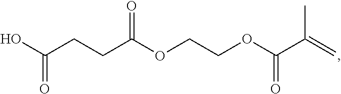 [SMILES: C=C(C)C(=O)OCCOC(=O)CCC(=O)O]

that is, MMES; the initiator in the mixed solution is SBP, and the structural formula of SBP is

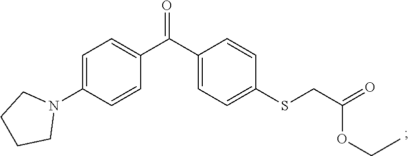 [SMILES: CCOC(=O)CSc1ccc(C(=O)c2ccc(N3CCCC3)cc2)cc1]

the structural formula of the quantum dot light-emitting layer formed by the initiator SBP and the ligand MMES is

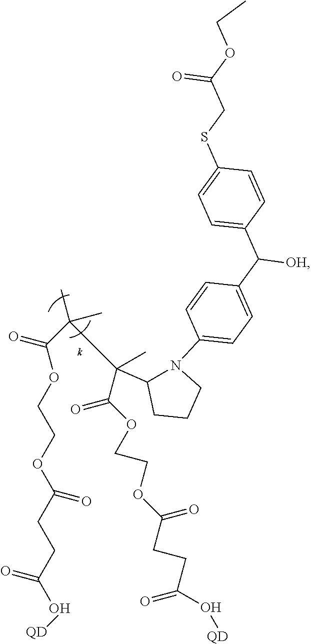

where QD represents a quantum dot, and k is a positive integer greater than or equal to 1.

FIG. 30 schematically illustrates the mechanism of the initiator SBP initiating the polymerization of ligands of the quantum dots. As illustrated in FIG. 30, the initiator SBP will turn into a more active

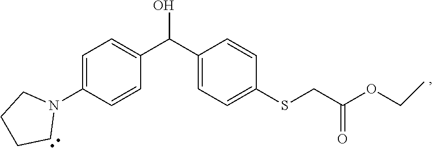 [SMILES: CCOC(=O)CSc1ccc(C(O)c2ccc(N3CCCC3)cc2)cc1]

which is abbreviated as SBP_{·}^{·}, under ultraviolet radiation. Under ultraviolet radiation, SBP_{·}^{·} attacks the carbon-carbon double bond of the adjacent ligand, causing the carbon-carbon double bond to break. When the carbon-carbon double bond is broken, two carbon free radicals (C^{·}) will be formed. One carbon free radical (C^{·}) combines with the amine free radical in the initiator SBP, and the other carbon free radical (C^{·}) further attacks the carbon-carbon double bond of the next adjacent ligand. When the carbon-carbon double bond of the next ligand is broken, the carbon free radicals (C^{·}) will also be formed, and the carbon free radical (C^{·}) will further attack the carbon-carbon double bond of the next adjacent ligand. After such continuous polymerization, a cross-link quantum dot light-emitting layer is finally formed. It should be noted that in FIG. 30, the structural formula of the formed cross-link quantum dot light-emitting layer is simplified as

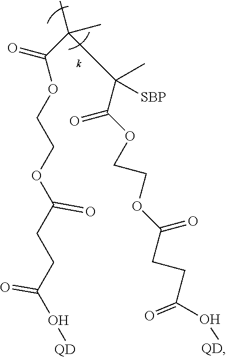

and this simplified structural formula represents the above structural formula

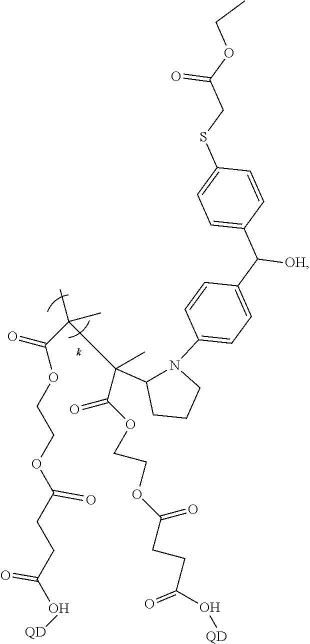

In some alternative embodiments, the structural formula of the ligand on the surface of the quantum dot in the mixed solution is

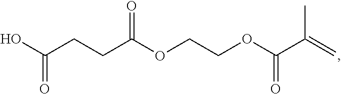 [SMILES: C=C(C)C(=O)OCCOC(=O)CCC(=O)O]

that is, MMES; the initiator in the mixed solution is (SBP)₂, and the structural formula of (SBP)₂ is

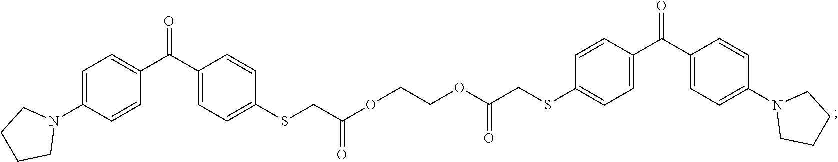 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]

the structural formula of the quantum dot light-emitting layer formed by the initiator (SBP)₂ and the ligand MMES is

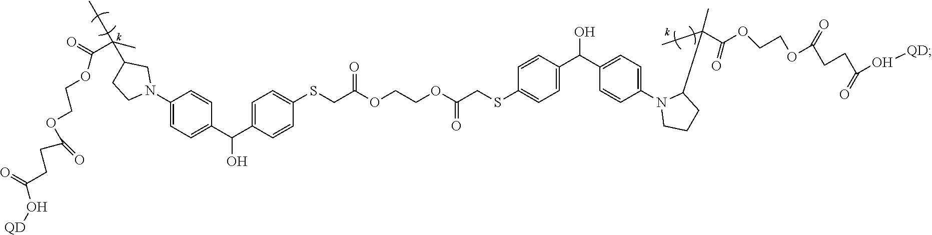

where QD represents a quantum dot, and k is a positive integer greater than or equal to 1.

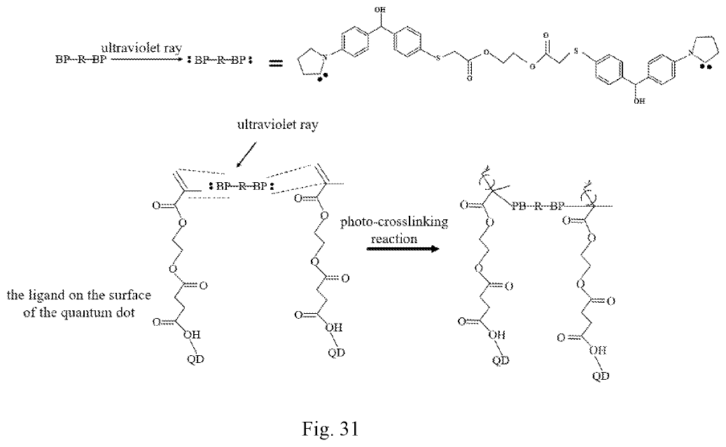

FIG. 31 schematically illustrates the mechanism of the initiator (SBP)₂ initiating the polymerization of ligands of the quantum dots. As illustrated in FIG. 31, the initiator (SBP)₂ will turn into a more active

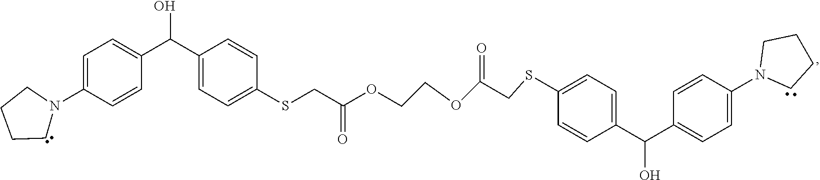 [SMILES: O=C(CSc1ccc(C(O)c2ccc(N3CCCC3)cc2)cc1)OCCOC(=O)CSc1ccc(C(O)c2ccc(N3CCCC3)cc2)cc1]

which is abbreviated as _{·}^{·}BP—R—BP_{·}^{·}, under ultraviolet radiation. Similar to the principle illustrated in FIG. 30, under ultraviolet radiation, _{·}^{·}BP—R—BP_{·}^{·} undergoes a polymerization reaction with the ligands on the surface of the quantum dots, and eventually a cross-link quantum dot light-emitting layer is formed. It should be noted that in FIG. 31, the structural formula of the formed cross-link quantum dot light-emitting layer is simplified as

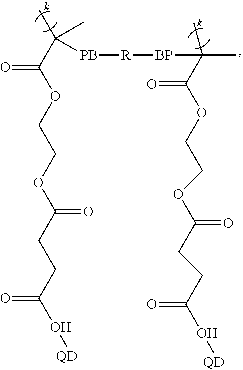

and this simplified structural formula represents the above structural formula

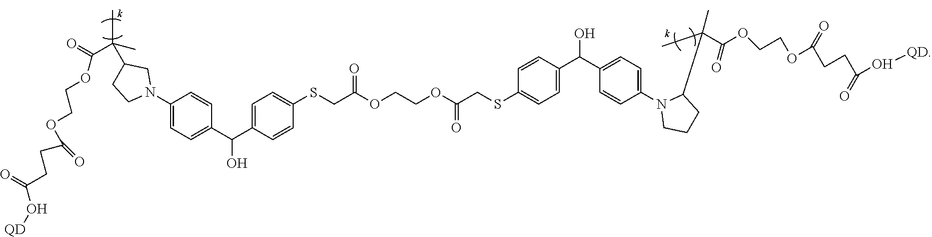

In some alternative embodiments, the structural formula of the ligand on the surface of the quantum dot in the mixed solution is

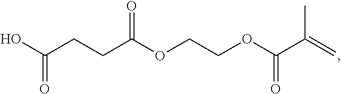 [SMILES: C=C(C)C(=O)OCCOC(=O)CCC(=O)O]

that is, MMES; the initiator in the mixed solution is (SBP)₃, and the structural formula of (SBP)₃ is

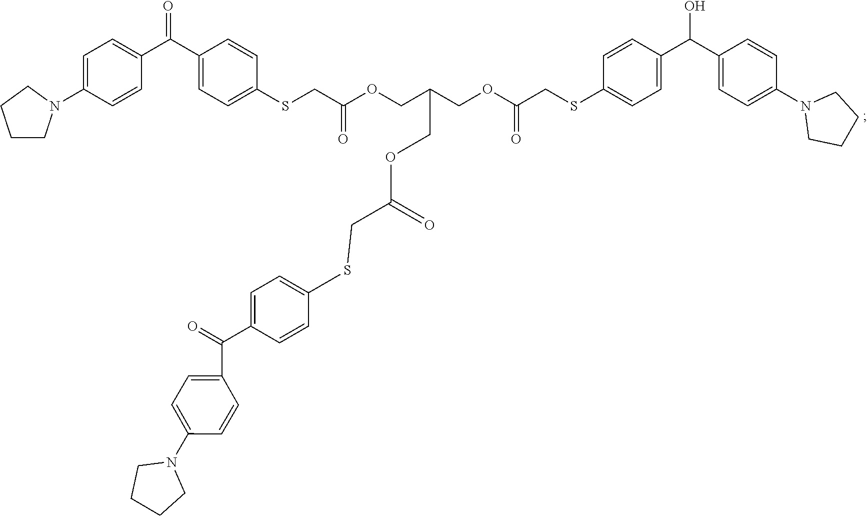 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(O)c2ccc(N3CCCC3)cc2)cc1]

the structural formula of the quantum dot light-emitting layer formed by the initiator (SBP) 3 and the ligand MMES is

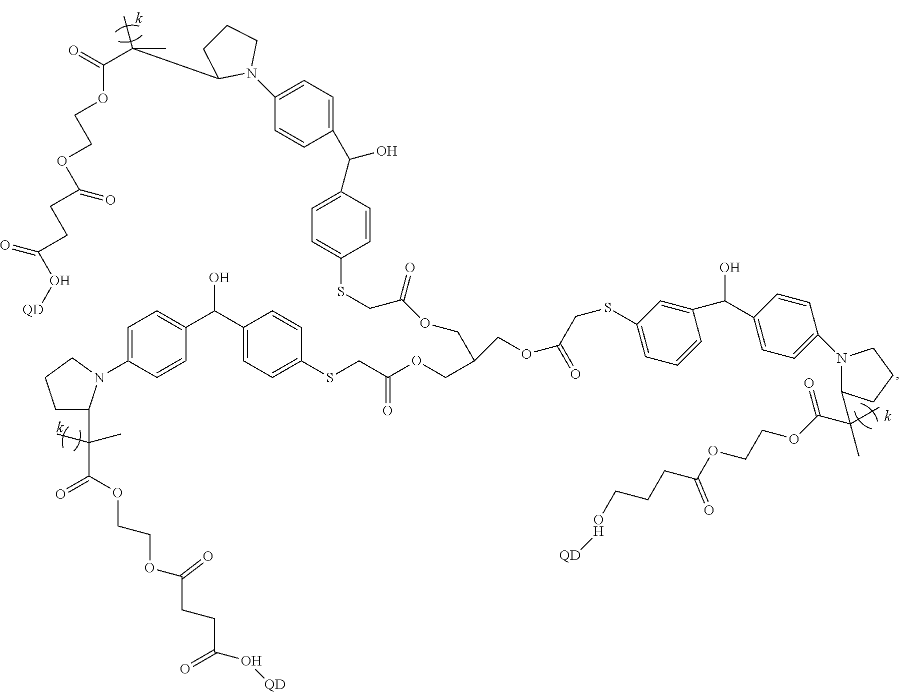

where QD represents a quantum dot, and k is a positive integer greater than or equal to 1.

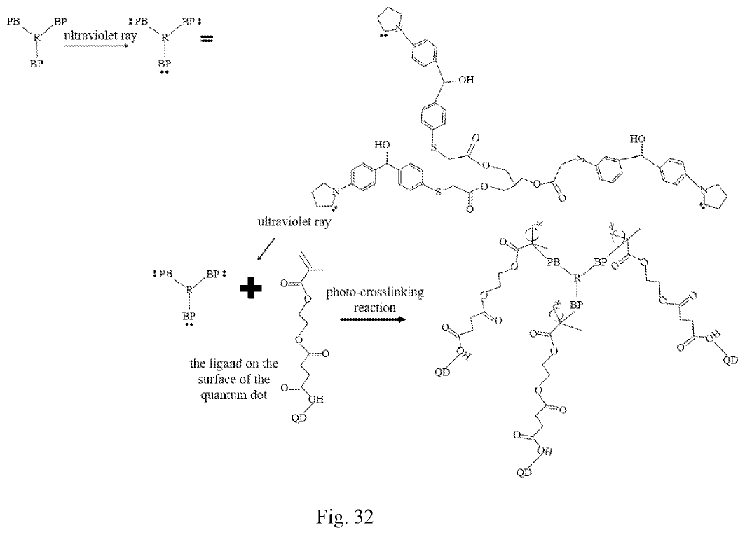

FIG. 32 schematically illustrates the mechanism of the initiator (SBP)₃ initiating the polymerization of ligands of the quantum dots. As illustrated in FIG. 32, the initiator (SBP)₃ will turn into a more active

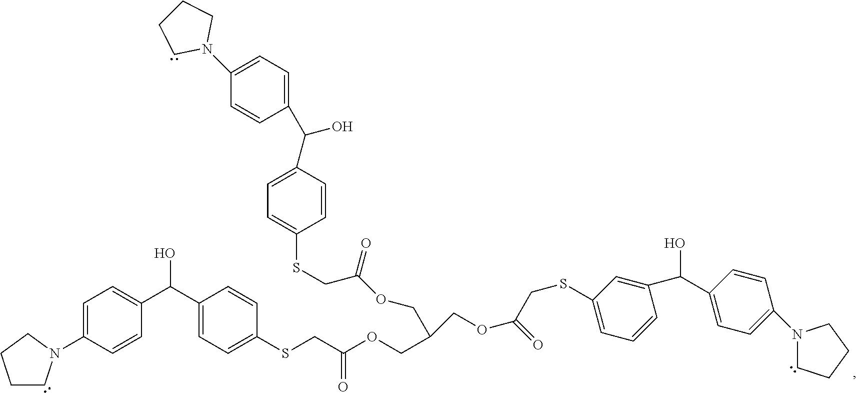

which is abbreviated as

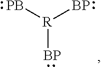

under ultraviolet radiation. Similar to the principle illustrated in FIG. 30, under ultraviolet radiation,

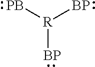

undergoes a polymerization reaction with the ligands on the surface of the quantum dots, and eventually a cross-link quantum dot light-emitting layer is formed. It should be noted that in FIG. 32, the structural formula of the formed cross-link quantum dot light-emitting layer is simplified as

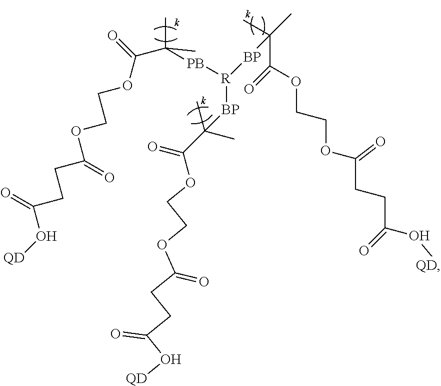

and this simplified structural formula represents the above structural formula

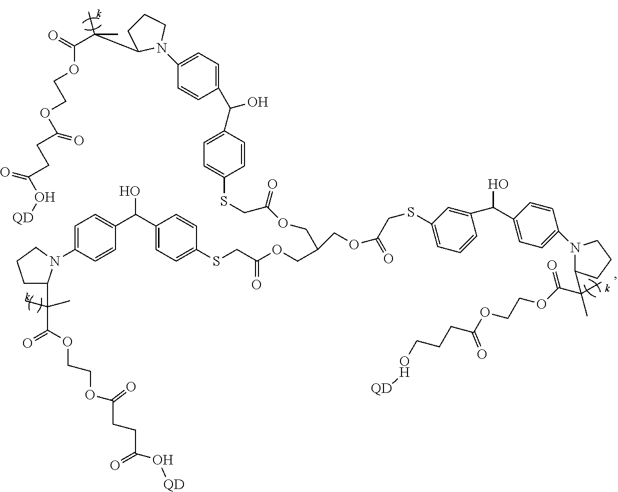

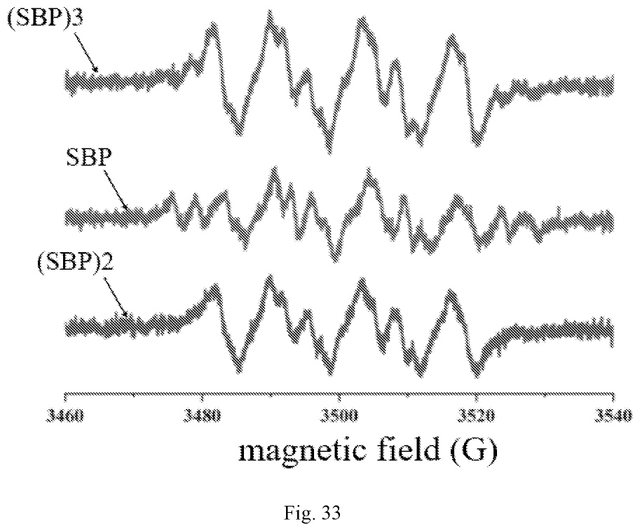

FIG. 33 further verifies the mechanism of quantum dot photo-patterning through the electron paramagnetic resonance spectrum. The electron paramagnetic resonance spectrum illustrates that the initiators SBP, (SBP)₂, and (SBP)₃ all produce strong free radical signal peaks, which illustrates that the main mechanism of quantum dot patterning is that the initiators SBP, (SBP)₂, and (SBP)₃ can initiate polymerization of free radicals of carbon-carbon double bonds of the ligands of the quantum dots.

The initiator with the best initiating effect can be selected from the initiators SBP, (SBP)₂, and (SBP)₃ based on some experimental data.

First, the effect of the exposure dose of the initiator on the film retention rate is explored. In the experiment, the ligand on the surface of the quantum dot can be MMES as mentioned above. The quantum dot with the ligand MMES has good dispersion in the PGMEA solvent (for example, the solubility is greater than 60 mg/mL). Initiators SBP, (SBP)₂, and (SBP)₃ also have good dispersion in the PGMEA solvent (for example, the solubility is about 5 mg/mL).

The hole transport material can be spin-coated on the substrate first. The concentration of the hole transport material is, for example, 8 mg/mL, and the spin-coating speed is about 2000 rpm. Then, three mixed solutions comprising quantum dots and initiators are respectively spin-coated on three substrates that are spin-coated with the hole transport materials. The quantum dots in the three mixed solutions are QD-MMES, and the initiators in the three mixed solutions are SBP, (SBP)₂, and (SBP)₃ respectively. The concentration of quantum dots QD-MMES is 25 mg/mL, the concentrations of initiators SBP, (SBP)₂, and (SBP)₃ are all 0.5 mg/mL, and the rotation speed of spin-coating is about 2000 rpm. The mixed solution solidifies into a film, and then the film is exposed.

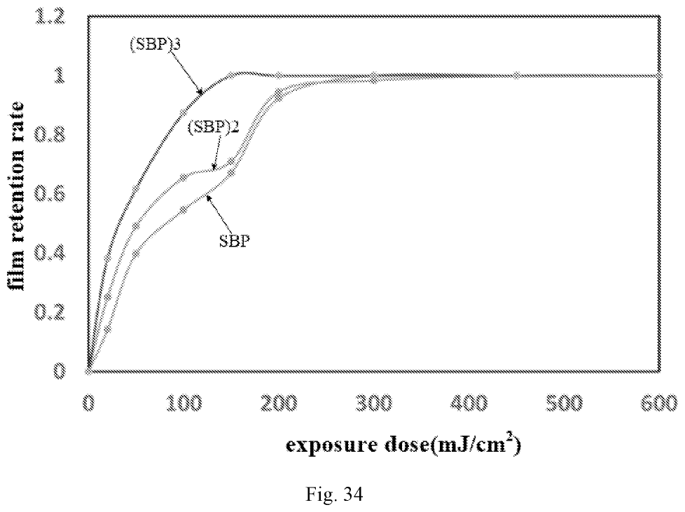

As illustrated in FIG. 34, the exposure times are 2 s, 5 s, 10 s, 15 s, 20 s, 30 s, 45 s, and 60 s respectively, the converted exposure doses are 20 mJ/cm², 50 mJ/cm², 100 mJ/cm², 150 mJ/cm², 200 mJ/cm², 300 mJ/cm², 450 mJ/cm², and 600 mJ/cm² respectively. As illustrated in FIG. 34, the initiators SBP and (SBP)₂ reach a film retention rate of 100% at an exposure dose of 300 mJ/cm², while the initiator (SBP)₃ reaches a film retention rate of 100% at an exposure dose of 150 mJ/cm², which illustrates that the initiator (SBP)₃ can achieve a film retention rate of 100% at a lower exposure dose and has better cross-link effect on quantum dots.

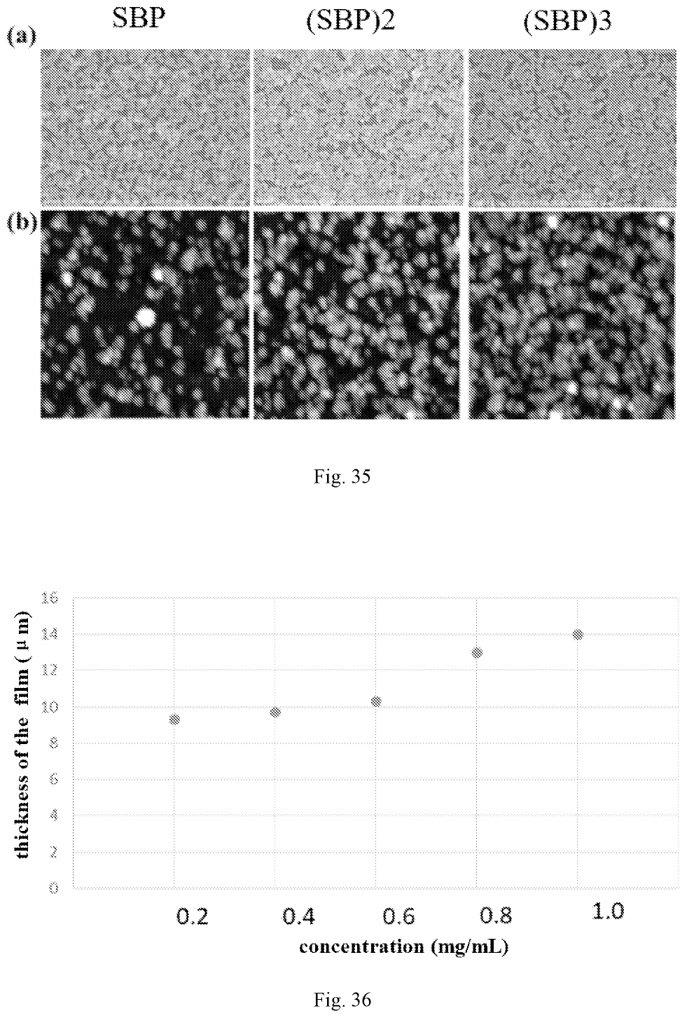

FIG. 35 illustrates scanning electron microscope pictures and atomic force microscope pictures of three kinds of quantum dot light-emitting layers after exposure and development. The initiators corresponding to the three kinds of quantum dot light-emitting layers are SBP, (SBP)₂, and (SBP)₃, and the corresponding exposure doses are all 150 mJ/cm². It can be seen from the scanning electron microscope pictures and atomic force microscope pictures in FIG. 35 that, three kinds of quantum dot light-emitting layers have good compactness and good film quality, this illustrates that the ligands of the quantum dots have undergone a good polymerization reaction and the initiators SBP, (SBP)₂, and (SBP)₃ are beneficial to forming a patterned quantum dot light-emitting layer with high quality. Further, the three kinds of quantum dot light-emitting layers are compared, compared with the quantum dot light-emitting layers comprising the initiators SBP and (SBP)₂, the quantum dot light-emitting layer comprising the initiator (SBP)₃ is the densest and has the best film quality after exposure and development.

FIG. 36 illustrates the relationship between the concentration of the initiator (SBP)₃ and the thickness of the quantum dot light-emitting layer. In this experiment, (SBP)₃ is selected as the initiator, the exposure dose is set to 150 mJ/cm², the concentration of the quantum dots is set to 25 mg/mL. The concentration of the initiator (SBP)₃ is changed to 0.2 mg/mL, 0.4 mg/mL, 0.6 mg/mL, 0.8 mg/mL, and 1.0 mg/mL, respectively. After exposure and development, a step meter is used to test the thickness of the quantum dot light-emitting layer. As illustrated in FIG. 36, as the concentration of the initiator (SBP) 3 increases, the thickness of the quantum dot light-emitting layer also gradually increases.

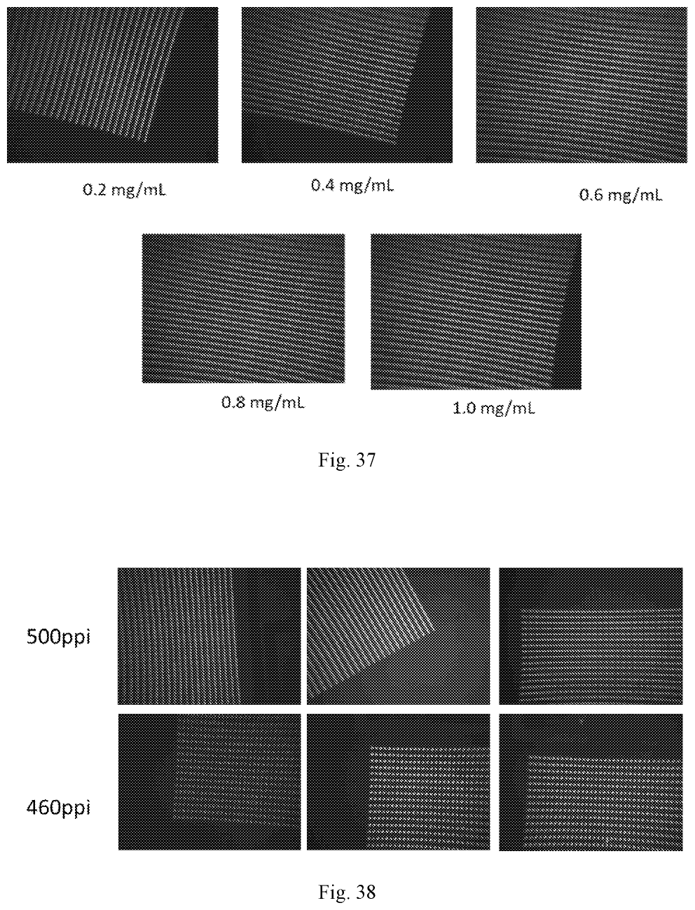

FIG. 37 illustrates the pictures of the quantum dot light-emitting layers under a fluorescence microscope after the mixed solution comprising different concentrations of initiators (SBP)₃ is cured to a film, exposed, and developed. It can be seen from FIG. 37 that the quantum dot light-emitting layers comprising different concentrations of initiators (SBP)₃ all have good patterning effects.

FIG. 38 illustrates pictures of patterned red, green and blue quantum dot light-emitting layers on the backplanes with different pixels per inch (PPI), wherein the middle picture represents the green quantum dot light-emitting layer, the picture on the left side of the middle picture represents the red quantum dot light-emitting layer, and the picture on the right side of the middle picture represents the blue quantum dot light-emitting layer. Taking the red quantum dot light-emitting layer as an example, the preparation process is roughly as follows: first, a solution comprising the hole transport material is spin-coated on the backplane, then a mixed solution including the red quantum dots and the initiator (SBP)₃ is spin-coated on the backplane and cured to form a film, then the film is exposed for 15 s and then developed with PGMEA, to form the patterned red quantum dot light-emitting layer. Using the same method, the patterned green quantum dot light-emitting layer and the patterned blue quantum dot light-emitting layer can be formed respectively. The pixels per inch of the backplane may be 460 ppi and 500 ppi respectively. In this way, as illustrated in FIG. 38, six different quantum dot light-emitting layers can be formed. It can be seen from FIG. 38 that the six quantum dot light-emitting layers all have good patterning effects, which illustrates that the initiator (SBP)₃ has universal applicability in different quantum dots (red, green, and blue quantum dots) and backplanes with different pixels per inch (460 ppi and 500 ppi).

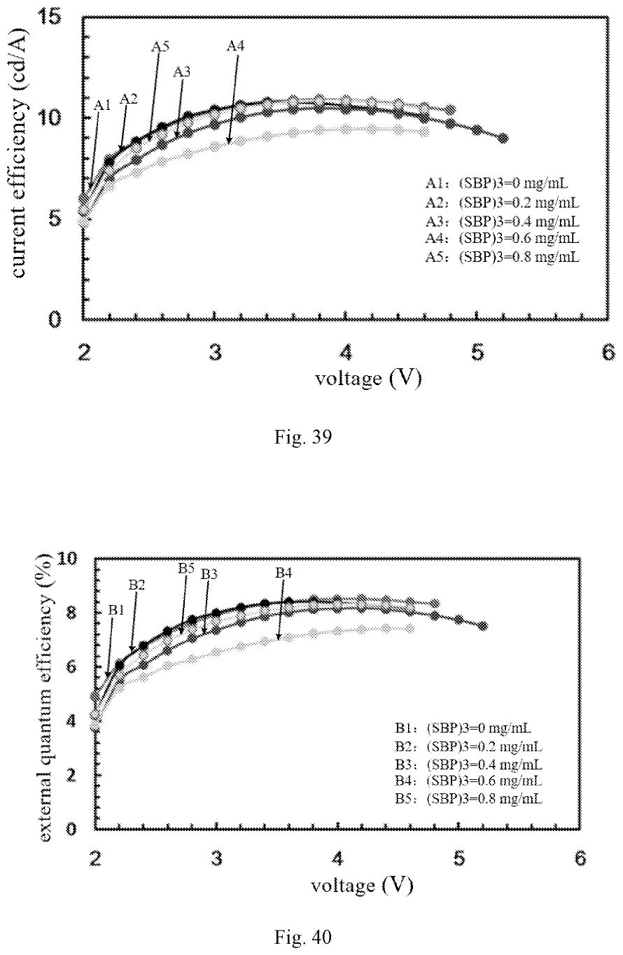

FIG. 39 explores the effect of the concentration of the initiator (SBP)₃ on the current efficiency of the light-emitting device, where the concentrations of the initiator (SBP)₃ are set to 0 mg/mL, 0.2 mg/mL, 0.4 mg/mL, 0.6 mg/mL, 0.8 mg/mL, respectively. As illustrated in FIG. 39, the curve A1 is a curve of the current efficiency of the light-emitting device varying with voltage when the concentration of the initiator (SBP)₃ is 0 mg/mL, which is a comparison curve; and the curves A2˜A5 are the curves of the current efficiency of the light-emitting device varying with voltage when the concentrations of the initiator (SBP)₃ are 0.2 mg/mL, 0.4 mg/mL, 0.6 mg/mL, and 0.8 mg/mL respectively. It can be seen from FIG. 39 that even if the concentration of the initiator increases to 0.8 mg/mL and the voltage increases from 2V to 6V, the current efficiency of the light-emitting device is basically the same as that of the comparison curve A1, which illustrates that the effect of the initiator (SBP)₃ on the current efficiency of quantum dot materials is very small or even negligible, and the initiator (SBP)₃ is an ideal initiator material.

FIG. 40 explores the effect of the concentration of the initiator (SBP)₃ on the external quantum efficiency of the light-emitting device, where the concentrations of the initiator (SBP)₃ are set to 0 mg/mL, 0.2 mg/mL, 0.4 mg/mL, 0.6 mg/mL, and 0.8 mg/mL respectively. As illustrated in FIG. 40, the curve B1 is a curve of the external quantum efficiency of the light-emitting device varying with voltage when the concentration of the initiator (SBP)₃ is 0 mg/mL, which is a comparison curve; and the curves B2˜B5 are the curves of the external quantum efficiency of the light-emitting device varying with voltage when the concentrations of the initiator (SBP)₃ are 0.2 mg/mL, 0.4 mg/mL, 0.6 mg/mL, and 0.8 mg/mL respectively. It can be seen from FIG. 40 that even if the concentration of the initiator increases to 0.8 mg/mL and the voltage increases from 2V to 6V, the external quantum efficiency of the light-emitting device is basically the same as that of the comparison curve B1, which illustrates that the effect of the initiator (SBP) 3 on the external quantum efficiency of quantum dot materials is very small or even negligible, and the initiator (SBP)₃ is an ideal initiator material.

It can be seen from the above data and pictures that the initiators SBP, (SBP)₂, and (SBP)₃ have good patterning effect on the quantum dot light-emitting layer, and can help to prepare the patterned quantum dot light-emitting layer with higher quality. Comparing the three initiators SBP, (SBP)₂, and (SBP)₃, the initiator (SBP)₃ has the best patterning effect on the quantum dot light-emitting layer.

According to another aspect of the present disclosure, a quantum dot light-emitting layer is provided. The quantum dot light-emitting layer comprises a plurality of quantum dots, at least some of the plurality of quantum dots have ligands on their surfaces, and the quantum dot light-emitting layer is generated by crosslinking the ligands on the surface of the quantum dots and the initiator. The ligand on the surface of the quantum dots may be the ligand described in any of the previous embodiments, and the initiator may be the initiator described in any of the previous embodiments.

The structural formula (1) of the initiator is

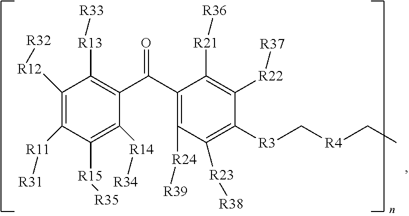

where R11, R12, R13, R14 and R15 may be same or different from each other, and R11, R12, R13, R14 and R15 may be respectively selected from any one of a hydrogen atom, a group comprising a nitrogen atom and a hydrogen atom, an ester group, and an alkyl group; R21, R22, R23 and R24 may be same or different from each other, and R21, R22, R23 and R24 may be respectively selected from any one of a hydrogen atom, a fluorine atom, a sulfur atom, an oxygen atom, an ester group, an alkyl group, and a group comprising a nitrogen atom and a hydrogen atom; R3 may be selected from any one of a sulfur atom, an oxygen atom, an ester group, an amide group, an alkyl group, a hydrogen atom, and a fluorine atom; R4 may be selected from any one of an ester group, an alkyl group, and a group comprising a nitrogen atom and a hydrogen atom; R31, R32, R33, R34, R35, R36, R37, R38 and R39 may be present or absent and may be same or different from each other, when at least one of R31, R32, R33, R34, R35, R36, R37, R38 and R39 is present, the at least one of R31, R32, R33, R34, R35, R36, R37, R38 and R39 may be selected from any one of an ester group, an alkyl group, and a group comprising a nitrogen atom and a hydrogen atom; n is a positive integer greater than or equal to 1, and the group of the initiator needs to comprise at least one of the sulfur atom, the oxygen atom, and the group comprising the nitrogen atom and the hydrogen atom.

The structural formula (4) of the ligand on the surface of the quantum dot is

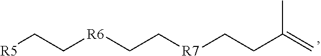

where R5 is coordinately connected to the quantum dot surface and is selected from any one of a sulfhydryl group, a carboxyl group and an amino group, R6 is selected from an ester group or an ether group, and R7 is selected from an ester group or an ether group.

Since the group of the initiator comprises at least one of the sulfur atom, the oxygen atom, the group comprising the nitrogen atom and the hydrogen atom, the activity and stability of free radicals can be improved, so that the initiator has high initiating activity, thereby increasing the initiating rate of the initiator, improving the degree of reaction between the quantum dots and the initiators in the exposure region per unit time, accelerating the polymerization reaction of the carbon-carbon double bonds of the ligands on the surface of the quantum dots, and helping to form a patterned quantum dot light-emitting layer with high quality.

In some embodiments, the structural formula (4) of the ligand on the surface of the quantum dot is specifically

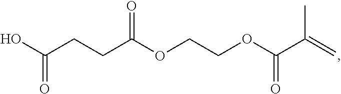 [SMILES: C=C(C)C(=O)OCCOC(=O)CCC(=O)O]

which is abbreviated as MMES; the structural formula (1) of the initiator is specifically

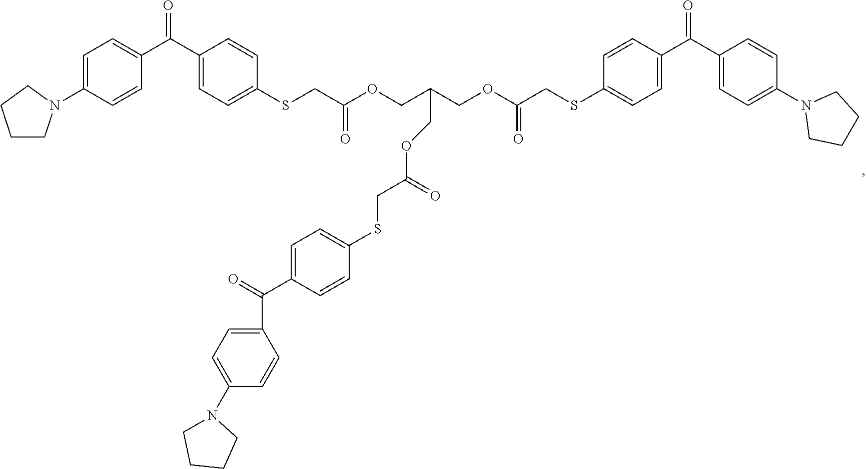 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(=O)c2ccc(N3CCCC3)cc2)cc1]

which is abbreviated as (SBP)₃; the structural formula of the quantum dot light-emitting layer generated by crosslinking the ligand MMES and the initiator (SBP)₃ is

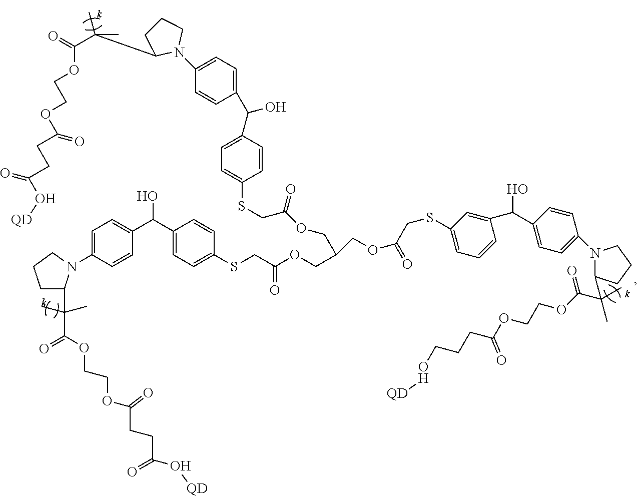

where QD represents a quantum dot, and k is a positive integer greater than or equal to 1.

The initiator (SBP)₃ has more initiating sites, so the initiating rate of the initiator (SBP)₃ per unit time is higher, resulting in a better cross-link effect of quantum dots.

In some embodiments, the structural formula (4) of the ligand on the surface of the quantum dot is specifically

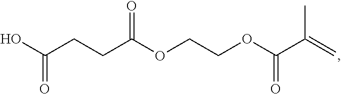 [SMILES: C=C(C)C(=O)OCCOC(=O)CCC(=O)O]

which is abbreviated as MMES; the structural formula (1) of the initiator is specifically

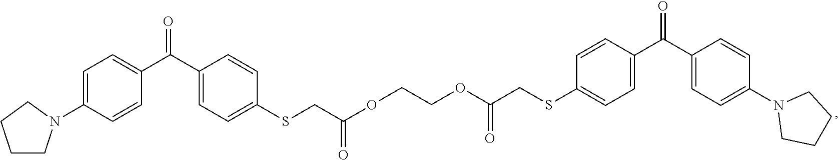 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]

which is abbreviated as (SBP)₂; the structural formula of the quantum dot light-emitting layer generated by crosslinking the ligand MMES and the initiator (SBP)₂ is

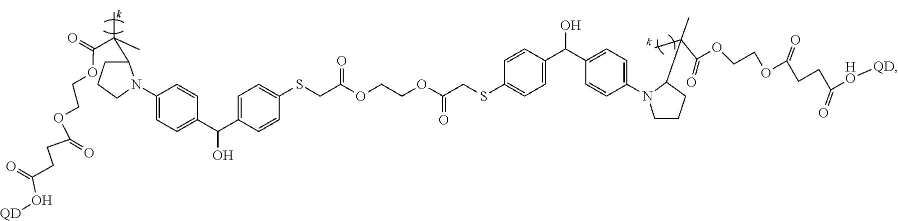

where QD represents a quantum dot, and k is a positive integer greater than or equal to 1.

The initiator (SBP)₂ also has more initiating sites, which can increase the initiating rate of the initiator per unit time, resulting in a better cross-link effect of quantum dots.

In some embodiments, the structural formula (4) of the ligand on the surface of the quantum dot is specifically

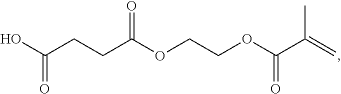 [SMILES: C=C(C)C(=O)OCCOC(=O)CCC(=O)O]

which is abbreviated as MMES; the structural formula (1) of the initiator is specifically

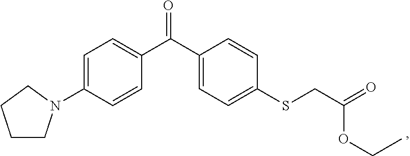 [SMILES: CCOC(=O)CSc1ccc(C(=O)c2ccc(N3CCCC3)cc2)cc1]

which is abbreviated as SBP; the structural formula of the quantum dot light-emitting layer generated by crosslinking the ligand MMES and the initiator SBP is

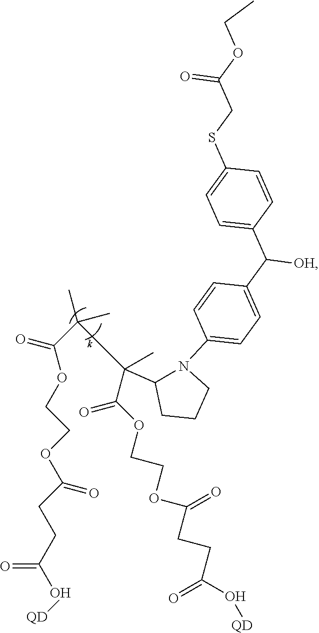

where QD represents a quantum dot, and k is a positive integer greater than or equal to 1.

Compared with the initiators (SBP)₂ and (SBP)₃, the initiator SBP has a simpler synthesis process and is therefore easier to obtain.

In the initiators SBP, (SBP)₂, and (SBP)₃, both the pyrrole group and the sulfur atom are introduced. In this way, on the one hand, under light radiation, the initiator can capture the hydrogen atom from the pyrrole group, turning the pyrrole group into an active amine free radical, the generated amine free radical can then react with oxygen to form a peroxide, the peroxide can capture a hydrogen atom and generate another amine free radical, while oxygen does not consume the triplet benzophenone structural unit in the entire process, so the introduction of the pyrrole group can eliminate the polymerization inhibition of the free radical polymerization reaction by oxygen in the system. Moreover, the active amine free radical has very little steric hindrance and high initiating activity, which can accelerate the polymerization reaction of the carbon-carbon double bonds of the ligands on the surface of the quantum dots. On the other hand, the thioether bond is broken under light radiation of the sulfur atom, generating the sulfur free radical, benzene free radical, etc., increasing the active centers of free radical. Moreover, the sulfur atom is a weak electron-donating group, which makes the overall molecular structure of the initiator appear as a push-pull-push conjugated system, causing the absorption of the entire molecule to be significantly red-shifted. Therefore, by simultaneously introducing the pyrrole group and the sulfur atom into the molecular structure, the activity and stability of free radicals can be greatly improved, thereby greatly increasing the initiating rate of the initiator, increasing the degree of reaction between the quantum dots and the initiators in the exposure region per unit time, helping to form a high-quality quantum dot pattern.

In some alternative embodiments, the structural formula of the ligand on the surface of the quantum dot is

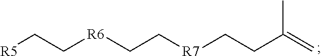

the initiator is a mixture of the benzophenone and the amine compound; and the structural formula of the quantum dot light-emitting layer generated by crosslinking the initiator with the quantum dots is

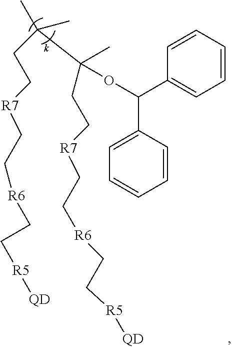

where R5 is selected from any one of a sulfhydryl group, a carboxyl group and an amino group, R6 is selected from an ester group or an ether group, R7 is selected from an ester group or an ether group, QD represents a quantum dot, and k is a positive integer greater than or equal to 1.

In some alternative embodiments, the structural formula of the ligand on the surface of the quantum dot is

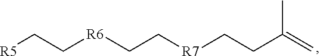

the initiator is a mixture of the benzophenone derivative and the amine compound. The structural formula of the benzophenone derivative is

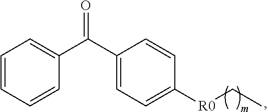

and the structural formula of the quantum dot light-emitting layer generated by crosslinking the initiator and the quantum dots is

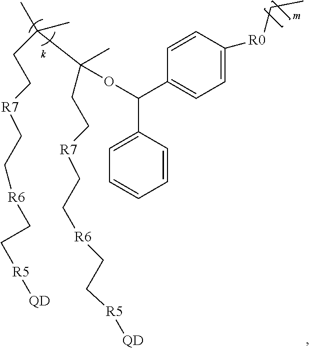

where R5 is selected from any one of a sulfhydryl group, a carboxyl group and an amino group, R6 is selected from an ester group or an ether group, R7 is selected from an ester group or an ether group, R0 is selected from any one of O, S, C₆H₅, OH, Br, Cl, and I, QD represents a quantum dot, k is a positive integer greater than or equal to 1, and m is a positive integer greater than or equal to 0.

In some embodiments, the patterned quantum dot light-emitting layer comprises a plurality of first quantum dot patterns, a plurality of second quantum dot patterns, and a plurality of third quantum dot patterns, the first quantum dot pattern may be used to emit red light, the second quantum dot pattern may be used to emit green light, and the third quantum dot pattern may be used to emit blue light.

The quantum dot light-emitting layer can be applied in the field of electro-light-emitting or photo-light-emitting.

In the field of electro-light-emitting, for example, the quantum dot light-emitting layer may be used as the light-emitting layer in a quantum dot light-emitting diode device. The electrons and holes generated in the device are transported to the quantum dot light-emitting layer under the electric field and recombine into excitons in the quantum dot light-emitting layer, resulting in energy level transition, thereby emitting light.

In the field of photo-light-emitting, the quantum dot light-emitting layer may be integrated into the backlight; alternatively, the quantum dot light-emitting layer may be integrated inside a panel, used in a liquid crystal display panel, used in an organic light-emitting diode display panel, or used in a sub-millimeter organic light-emitting diode display panel, as a color film; alternatively, the quantum dot light-emitting layer may be disposed on the light-emitting side of an organic light-emitting diode display panel that emits blue light, and emits red, green, and blue light under the excitation of blue light, thereby achieving full-color display.

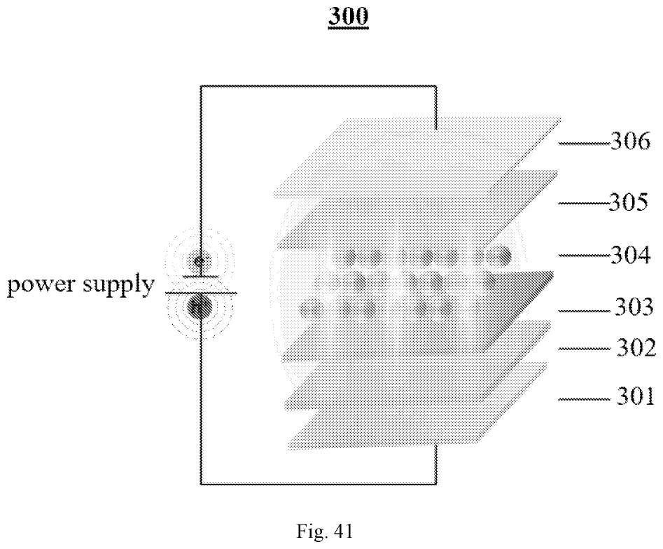

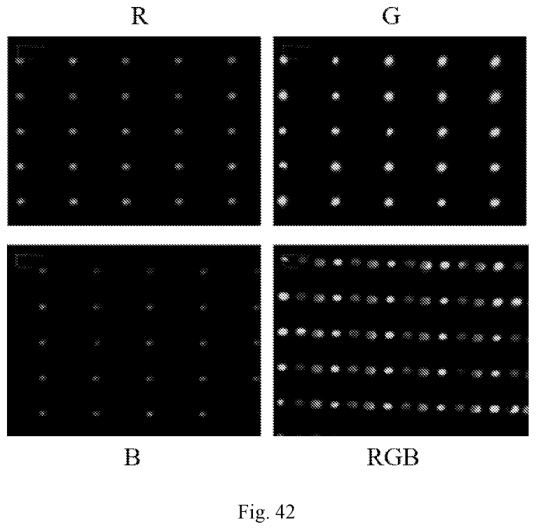

FIG. 41 illustrates a schematic structural diagram of a light-emitting device 300. As illustrated in FIG. 41, the light-emitting device 300 comprises: a first electrode layer 301; a hole injection layer 302 located on the first electrode layer 301; a hole transport layer 303 located on the side of the hole injection layer 302 away from the first electrode layer 301; a quantum dot light-emitting layer 304 located on the side of the hole transport layer 303 away from the first electrode layer 301, the quantum dot light-emitting layer 304 may be the quantum dot light-emitting layer described in any of the previous embodiments; an electron transport layer 305 located on the side of the quantum dot light-emitting layer 304 away from the first electrode layer 301; and a second electrode layer 306 located on the side of the electron transport layer 305 away from the first electrode layer 301.

The light-emitting device 300 is an electro-light-emitting device, and its light-emitting principle is that, the quantum dot light-emitting layer 304 is sandwiched between the first electrode 301 and the second electrode 306, the power supply is provided to the first electrode 301 and the second electrode 306 respectively, electrons and holes are generated under the action of the electric field and are transported to the quantum dot light-emitting layer 304 and recombine into excitons in the quantum dot light-emitting layer 304, resulting in an energy level transition, thereby emitting light. The light-emitting device 300 has advantages such as high color purity, high contrast, and high stability.

In some embodiments, the first electrode 301 is an anode, and the second electrode 306 is a cathode. In this case, the light-emitting device 300 has an upright structure. The material of the first electrode 301 may be ITO, and the material of the second electrode 306 may be Al.

In some alternative embodiments, the light-emitting device 300 may also have an inverted structure. In this case, the stacking relationship of the layers of the light-emitting device 300 is: a cathode, an electron transport layer located on the cathode; a quantum dot light-emitting layer located on the side of the electron transport layer away from the cathode; a hole transport layer located on the side of the quantum dot light-emitting layer away from the cathode; a hole injection layer located on the side of the hole transport layer away from the cathode; and an anode located on the side of the hole injection layer away from the cathode.

The light-emitting device 300 may be of a top emission type or a bottom emission type.

FIG. 42 illustrates four fluorescence microscopy pictures, where R represents the patterning effect of the red quantum dot light-emitting layer on the 460 ppi backplane, G represents the patterning effect of the green quantum dot light-emitting layer on the 460 ppi backplane, G represents the patterning effect of the blue quantum dot light-emitting layer on the 460 ppi backplane, and RGB represents the patterning effect of the quantum dot light-emitting layer comprising red, green, and blue quantum dots on the 460 ppi backplane. The initiator in the quantum dot light-emitting layer may be SBP, or (SBP)₂, or (SBP)₃. It can be seen from FIG. 42 that both the single-color quantum dot light-emitting layer and the quantum dot light-emitting layer comprising red, green, and blue quantum dots have good patterning effects.

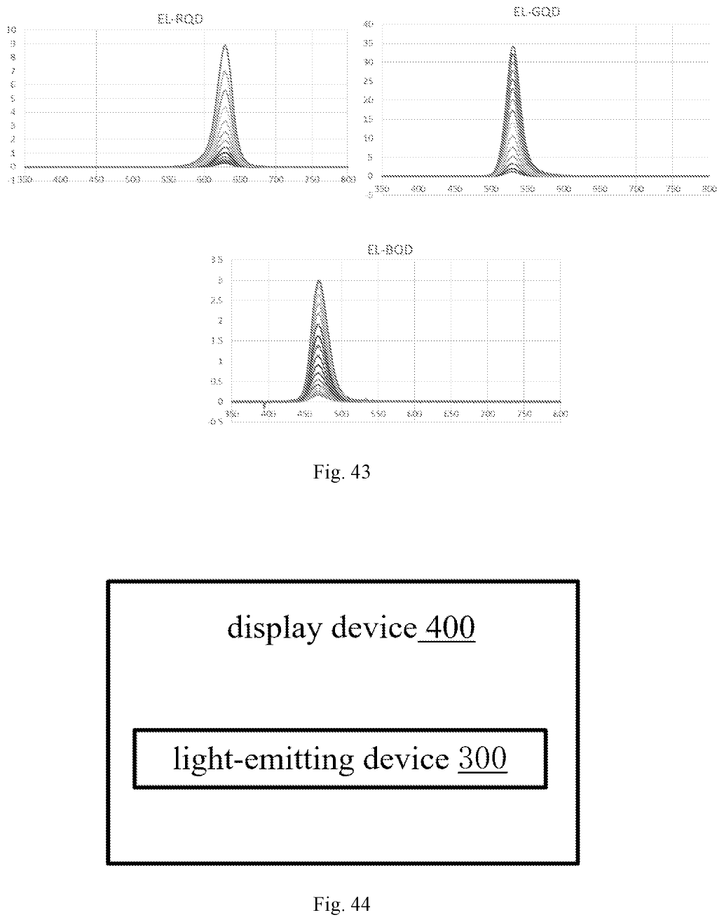

FIG. 43 illustrates three spectra, wherein EL-RQD represents the spectrum of a light-emitting device comprising a red quantum dot light-emitting layer under electrical excitation, EL-GQD represents the spectrum of a light-emitting device comprising a green quantum dot light-emitting layer under electrical excitation, and EL-BQD represents the spectrum of a light-emitting device comprising a blue quantum dot light-emitting layer under electrical excitation. As illustrates in FIG. 43, as the voltage increases, the three spectra only illustrate the light-emitting peak of single-color quantum dots without crosstalk from other colors, which indicates that overlaying of full-color quantum dots is achieved.

For other technical effects of the light-emitting device 300, reference may be made to the technical effects of the quantum dot light-emitting layer described in the previous embodiments. For the sake of simplicity, the technical effects of the light-emitting device 300 will not be repeated here.

FIG. 44 illustrates a block diagram of a display device 400. The display device 400 comprises a plurality of sub-pixels, and each sub-pixel is provided with a light-emitting device 300. At least two light-emitting devices 300 among the plurality of light emitting devices 300 emit light of different colors. For example, some of the plurality of light-emitting devices 300 comprise a red quantum dot light-emitting layer and are used to emit red light; some of the plurality of light-emitting devices 300 comprise a green quantum dot light-emitting layer and are used to emit green light; some of the plurality of light-emitting devices 300 comprise a blue quantum dot light-emitting layer and are used to emit blue light, thereby enabling the display device 400 to achieve full-color display.

The display device 400 may be any product or component that displays based on quantum dots, such as a mobile phone, a tablet computer, a television, a monitor, a notebook computer, a digital photo frame, a navigator, and the like.

The display device 400 may have substantially the same technical effect as the light-emitting device 300 described in the previous embodiment. For the sake of simplicity, the technical effect of the display device 400 will not be repeated here.

It will be understood that although the terms first, second, third, etc. may be used herein to describe various elements, components, regions, layers and/or portions, these elements, components, regions, layers and/or portions should not be limited by these terms. These terms are only used to distinguish an element, component, region, layer or portion from another element, component, region, layer or portion. Thus, a first element, component, region, layer or portion discussed above could be termed a second element, component, region, layer or portion without departing from the teachings of the present disclosure.

Spatially relative terms such as “row”, “column”, “below”, “above”, “left”, “right”, etc. may be used herein for ease of description to describe factors such as the relationship of an element or feature to another element(s) or feature(s) illustrated in the figures. It will be understood that these spatially relative terms are intended to encompass different orientations of the device in use or operation in addition to the orientation depicted in the figures. For example, if the device in the figures is turned over, elements described as “below” other elements or features would then be oriented “above” other elements or features. Thus, the exemplary term “below” can encompass both an orientation of above and below. The device may be otherwise oriented (rotated 90 degrees or at other orientations) and the spatially relative descriptors used herein are interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to limit the present disclosure. As used herein, the singular forms “a,” “an,” and “the” are intended to comprise the plural forms as well, unless the context clearly dictates otherwise. It will be further understood that the terms “comprise” and/or “include” when used in this specification designate the presence of stated features, integers, steps, operations, elements and/or parts, but do not exclude the presence or addition of one or more other features, integers, steps, operations, elements, components and/or groups thereof. As used herein, the term “and/or” comprises any and all combinations of one or more of the associated listed items. In the description of this specification, description with reference to the terms “an embodiment,” “another embodiment,” etc. means that a particular feature, structure, material, or characteristic described in connection with the embodiment is included in at least one embodiment of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples as well as the features of the different embodiments or examples described in this specification without conflicting each other.

It will be understood that when an element or layer is referred to as being “on”, “connected to”, “coupled to”, or “adjacent to” another element or layer, it may be directly on, directly connected to, directly coupled to, or directly adjacent to another element or layer, or intervening elements or layers may be present. In contrast, when an element is referred to as being “directly on”, “directly connected to”, “directly coupled to”, “directly adjacent to” another element or layer, with no intervening elements or layers present. However, in no case should “on” or “directly on” be interpreted as requiring a layer to completely cover the layer below.

Embodiments of the disclosure are described herein with reference to schematic illustrations (and intermediate structures) of idealized embodiments of the disclosure. As such, variations to the shapes of the illustrations are to be expected, e.g., as a result of manufacturing techniques and/or tolerances. Accordingly, embodiments of the present disclosure should not be construed as limited to the particular shapes of the regions illustrated herein, but are to comprise deviations in shapes due, for example, to manufacturing. Thus, the regions illustrated in the figures are schematic in nature and their shapes are not intended to illustrate the actual shape of a region of a device and are not intended to limit the scope of the present disclosure.

Unless otherwise defined, all terms (comprising technical and scientific terms) used herein have the same meaning as commonly understood by one of ordinary skill in the art to which this disclosure belongs. It will be further understood that terms such as those defined in commonly used dictionaries should be construed to have meanings consistent with their meanings in the relevant art and/or the context of this specification, and will not be idealized or overly interpreted in a formal sense, unless expressly defined as such herein.

As will be appreciated by those skilled in the art, although the steps of the methods of the present disclosure are depicted in a particular order in the figures, this does not require or imply that the steps must be performed in that particular order, unless the context clearly dictates otherwise. Additionally or alternatively, multiple steps may be combined into one step for execution, and/or one step may be decomposed into multiple steps for execution. Furthermore, other method steps may be inserted between the steps. The inserted steps may represent such as improvements of a method described herein, or may be unrelated to the method. Also, a given step may not be fully complete before the next step starts.

The above descriptions are merely specific embodiments of the present disclosure, but the protection scope of the present disclosure is not limited thereto. Any changes or substitutions that those skilled in the art can easily think of within the technical scope disclosed by the present disclosure, should be comprised within the protection scope of the present disclosure. Therefore, the protection scope of the present disclosure should be based on the protection scope of the claims.

1. An initiator having a structural formula 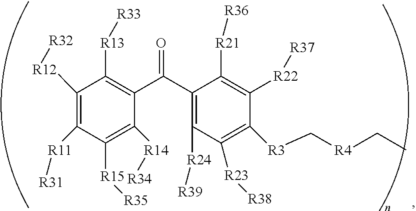
where
R11, R12, R13, R14 and R15 are same or different from each other, and R11, R12, R13, R14 and R15 are respectively selected from any one of a hydrogen atom, a group comprising a nitrogen atom and a hydrogen atom, an ester group, and an alkyl group;
R21, R22, R23 and R24 are same or different from each other, and R21, R22, R23 and R24 are respectively selected from any one of a hydrogen atom, a fluorine atom, a sulfur atom, an oxygen atom, an ester group, an alkyl group, and a group comprising a nitrogen atom and a hydrogen atom;
R3 is selected from any one of a sulfur atom, an oxygen atom, an ester group, an amide group, an alkyl group, a hydrogen atom, and a fluorine atom;
R4 is selected from any one of an ester group, an alkyl group, and a group comprising a nitrogen atom and a hydrogen atom;
R31, R32, R33, R34, R35, R36, R37, R38 and R39 are present or absent and are same or different from each other, when at least one of R31, R32, R33, R34, R35, R36, R37, R38 and R39 is present, the at least one of R31, R32, R33, R34, R35, R36, R37, R38 and R39 is selected from any one of an ester group, an alkyl group, and a group comprising a nitrogen atom and a hydrogen atom; and
n is a positive integer greater than or equal to 1, a group of the initiator comprises at least one of the sulfur atom, the oxygen atom, and the group comprising the nitrogen atom and the hydrogen atom, the group of the initiator refers to a group except a benzophenone matrix in the structural formula of the initiator.

2. The initiator according to claim 1, wherein R31, R32, R33, R34, R35, R36, R37, R38 and R39 are absent, R11, R12, R13, R14 and R15 are respectively selected from the hydrogen atom or the group comprising the nitrogen atom and the hydrogen atom, R21, R22, R23 and R24 are respectively selected from the hydrogen atom or the fluorine atom, and R3 is selected from any one of the sulfur atom, the oxygen atom, the ester group, the amide group, and the alkyl group.

3. The initiator according to claim 1, wherein the group comprising the nitrogen atom and the hydrogen atom is a pyrrole group or a tertiary amine group.

4. The initiator according to claim 1, wherein the structural formula of the initiator is

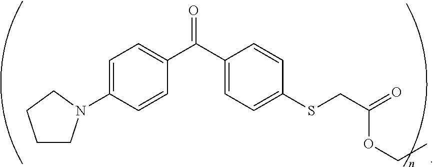

5. The initiator according to claim 4, wherein,
n=3, the structural formula of the initiator is 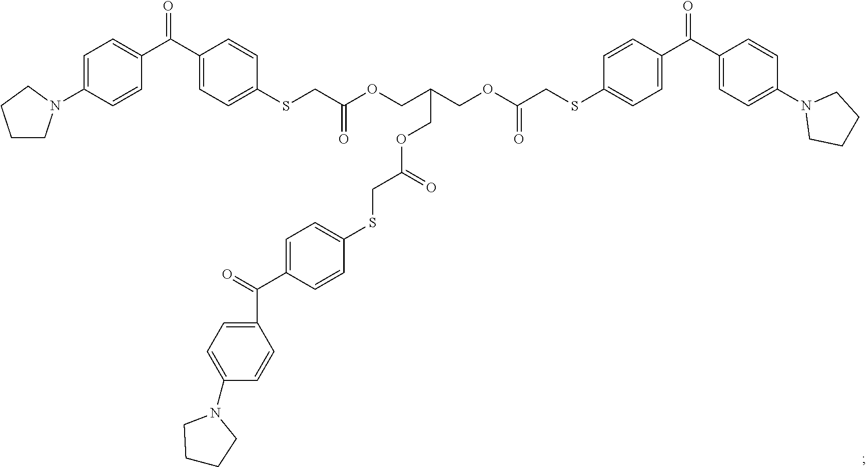 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(=O)c2ccc(N3CCCC3)cc2)cc1]
or,
n=2, the structural formula of the initiator is 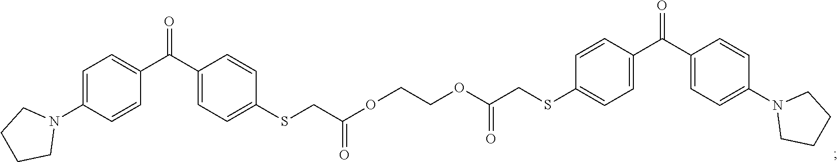 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]
or,
n=4, the structural formula of the initiator is 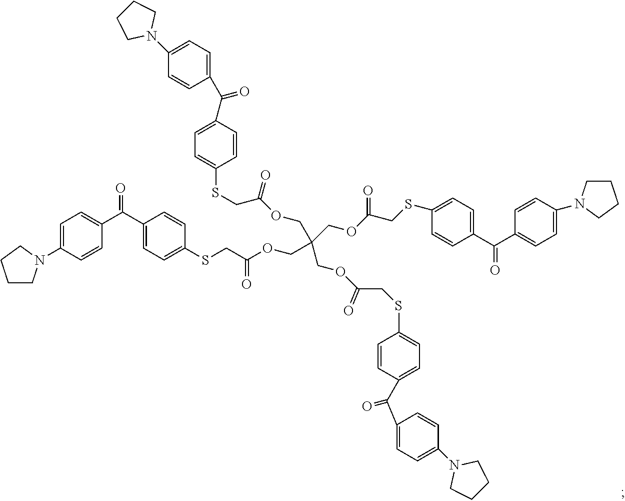 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)(COC(=O)CSc1ccc(C(=O)c2ccc(N3CCCC3)cc2)cc1)COC(=O)CSc1ccc(C(=O)c2ccc(N3CCCC3)cc2)cc1]
or,
n=1, the structural formula of the initiator is

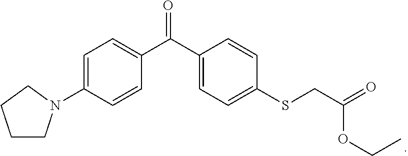 [SMILES: CCOC(=O)CSc1ccc(C(=O)c2ccc(N3CCCC3)cc2)cc1]

6. The initiator according to claim 1, wherein the structural formula of the initiator is 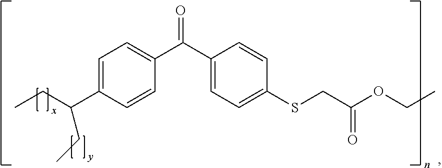
where x and y are both positive integers greater than or equal to 1.

7. The initiator according to claim 6, wherein a number of carbon atoms in a branch chain connected to the nitrogen atom is 2˜30.

8. An initiator being a mixture of a benzophenone and an amine compound or a mixture of a benzophenone derivative and an amine compound,
wherein a structural formula of the benzophenone derivative is 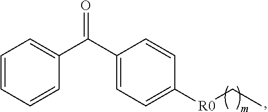
R0 is selected from any one of O, S, C₆H₅, OH, Br, Cl, and I, and m is a positive integer greater than or equal to 0.

9. The initiator according to claim 8, wherein the amine compound is a pyrrole or a tertiary amine.

10. A quantum dot, wherein a surface of the quantum dot has a ligand, and a structural formula of the ligand is 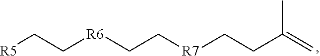
where R5 is coordinately connected to the surface of the quantum dot and is selected from any one of a sulfhydryl group, a carboxyl group and an amino group, R6 is selected from an ester group or an ether group, R7 is selected from an ester group or an ether group, the ligand is configured to undergo a photosensitive reaction under light radiation with the initiator according to claim 1.

11. The quantum dot according to claim 10, wherein the structural formula of the ligand is

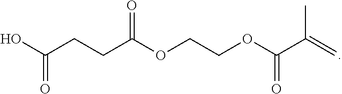 [SMILES: C=C(C)C(=O)OCCOC(=O)CCC(=O)O]

12. A quantum dot light-emitting layer comprising a plurality of quantum dots, wherein a surface of at least some of the plurality of quantum dots has a ligand, and the quantum dot light-emitting layer is generated by crosslinking the quantum dots having the ligands with the initiator according to claim 1.

13. The quantum dot light-emitting layer according to claim 12, wherein
a structural formula of the ligand is 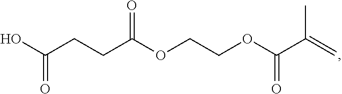 [SMILES: C=C(C)C(=O)OCCOC(=O)CCC(=O)O]
the structural formula of the initiator is 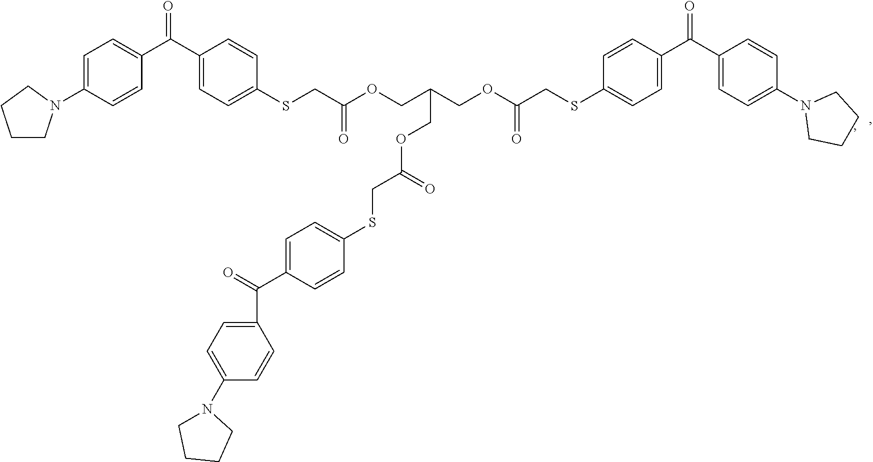 [SMILES: O=C(CSc1ccc(C(=O)c2ccc(N3CCCC3)cc2)cc1)OCC(COC(=O)CSc1ccc(C(=O)c2ccc(N3CCCC3)cc2)cc1)COC(=O)CSc1ccc(C(=O)c2ccc(N3CCCC3)cc2)cc1]
and a structural formula of the quantum dot light-emitting layer is 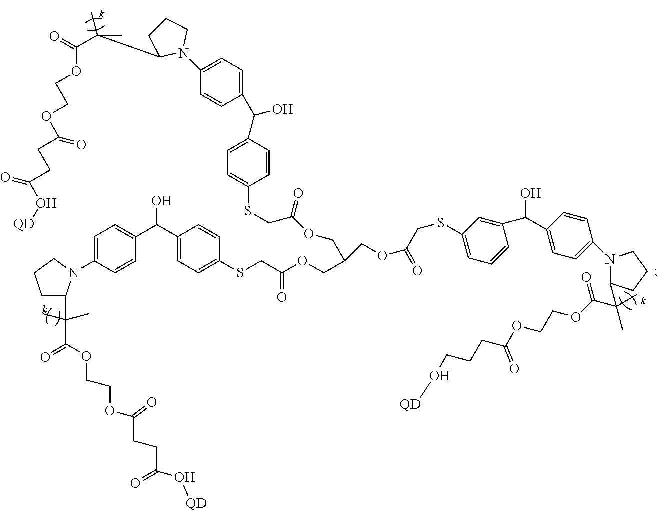
or,
a structural formula of the ligand is 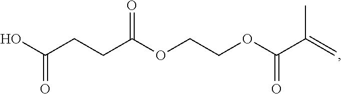 [SMILES: C=C(C)C(=O)OCCOC(=O)CCC(=O)O]
the structural formula of the initiator is 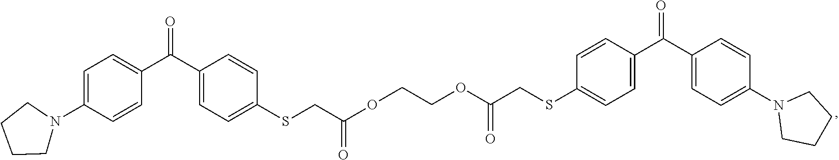 [SMILES: O=C(CSc1ccc(C(=O)c2ccc(N3CCCC3)cc2)cc1)OCCOC(=O)CSc1ccc(C(=O)c2ccc(N3CCCC3)cc2)cc1]
and a structural formula of the quantum dot light-emitting layer is 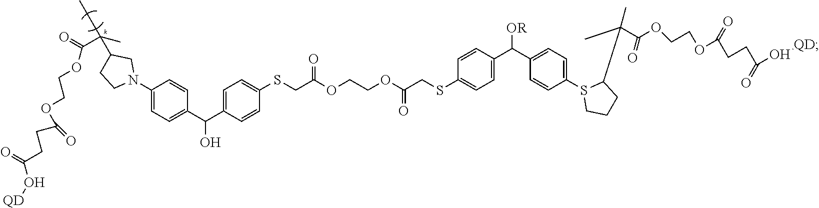
or,
a structural formula of the ligand is 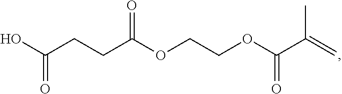 [SMILES: C=C(C)C(=O)OCCOC(=O)CCC(=O)O]
the structural formula of the initiator is 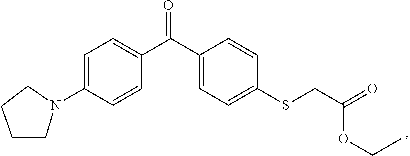 [SMILES: CCOC(=O)CSc1ccc(C(=O)c2ccc(N3CCCC3)cc2)cc1]
and a structural formula of the quantum dot light-emitting layer is 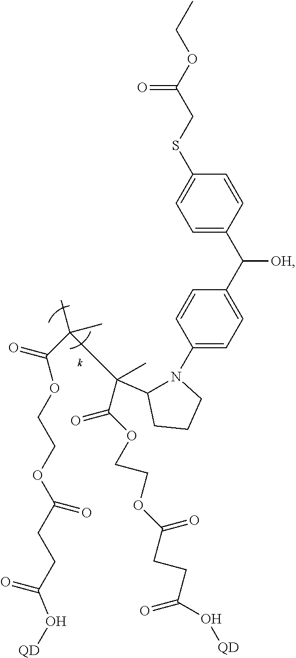
where QD represents a quantum dot, and k is a positive integer greater than or equal to 1.

14. The quantum dot light-emitting layer according to claim 12, wherein
a structural formula of the ligand is 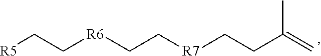
the initiator is a mixture of a benzophenone and an amine compound, and a structural formula of the quantum dot light-emitting layer is 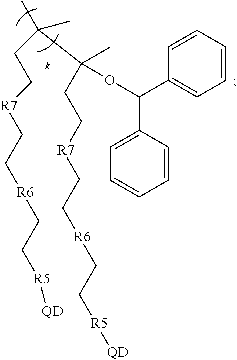
or,
a structural formula of the ligand is 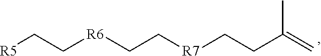
the initiator is a mixture of a benzophenone derivative and an amine compound, a structural formula of the benzophenone derivative is 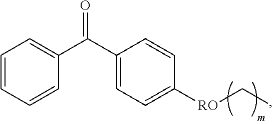
and a structural formula of the quantum dot light-emitting layer is 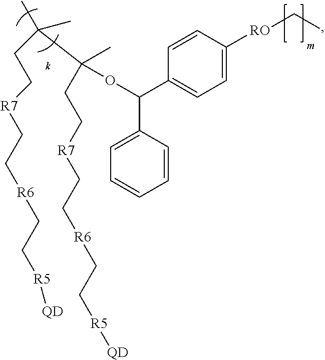
where R5 is selected from any one of a sulfhydryl group, a carboxyl group and an amino group, R6 is selected from an ester group or an ether group, R7 is selected from an ester group or an ether group, R0 is selected from any one of O, S, C₆H₅, OH, Br, Cl and I, QD represents a quantum dot, k is a positive integer greater than or equal to 1, and m is a positive integer greater than or equal to 0.

15. The quantum dot light-emitting layer according to claim 12, wherein the quantum dot light-emitting layer comprises a plurality of first quantum dot patterns, a plurality of second quantum dot patterns, and a plurality of third quantum dot patterns, the first quantum dot patterns are configured to emit red light, the second quantum dot patterns are configured to emit green light, and the third quantum dot patterns are configured to emit blue light.

16. A light-emitting device comprising:
a first electrode layer;
a hole injection layer on the first electrode layer;
a hole transport layer on a side of the hole injection layer away from the first electrode layer;
the quantum dot light-emitting layer according to claim 12, the quantum dot light-emitting layer being on a side of the hole transport layer away from the first electrode layer;
an electron transport layer on a side of the quantum dot light-emitting layer away from the first electrode layer; and
a second electrode layer on a side of the electron transport layer away from the first electrode layer.

17. A display device comprising a plurality of light-emitting devices according to claim 16, wherein at least two of the plurality of light-emitting devices are configured to emit light of different colors.

18. A method of manufacturing a patterned quantum dot light-emitting layer, comprising:
providing a substrate;
applying a mixed solution on the substrate, the mixed solution comprising a quantum dot and an initiator, a surface of the quantum dot having a ligand comprising a carbon-carbon double bond, and the initiator being the initiator according to claim 1; and
initiating a polymerization reaction of the carbon-carbon double bonds of the ligand by the initiator under light radiation to form the patterned quantum dot light-emitting layer.

19. The method according to claim 18, further comprising curing the mixed solution applied on the substrate to form an intermediate layer,
wherein the initiating the polymerization reaction of the carbon-carbon double bond of the ligand by the initiator under light radiation to form the patterned quantum dot light-emitting layer, comprises:
exposing the intermediate layer by using a mask, allowing ultraviolet light to pass through the mask to expose the intermediate layer, and initiating the polymerization reaction of the carbon-carbon double bond of the ligand by the initiator under the ultraviolet light; and
developing a polymerized intermediate layer by using a developer, and dissolving an unexposed portion of the polymerized intermediate layer by the developer to form the patterned quantum dot light-emitting layer.

20. The method according to claim 18, wherein a concentration of quantum dots in the mixed solution is about 25˜30 mg/mL, and a concentration of the initiator in the mixed solution is about 0.1˜1.0 mg/mL.
